(19) United States
(12) Patent Application Publication
(10) Pub. No.: US 2020/0232010 A1
(43) Pub. Date: Jul. 23, 2020

(54) METHODS, COMPOSITIONS, AND SYSTEMS FOR IMPROVING RECOVERY OF NUCLEIC ACID MOLECULES

(71) Applicant: GUARDANT HEALTH, INC., Redwood City, CA (US)

(72) Inventors: Stefanie Ann Ward MORTIMER, Morgan Hill, CA (US); Dustin Howard HITE, Redwood City, CA (US); Laura Michelle MELROY, San Francisco, CA (US)

(21) Appl. No.: 16/724,045

(22) Filed: Dec. 20, 2019

Related U.S. Application Data
(60) Provisional application No. 62/783,046, filed on Dec. 20, 2018.

(51) Int. Cl. C12Q 1/686

(52) U.S. Cl. CPC C12Q 1/686; C12Q 2565/514; C12Q 2600/156

(57) ABSTRACT

In an aspect, the present disclosure provides a method for analyzing nucleic acid molecules in a sample of polynucleotides, comprising: (a) adding a subset of fragment size control molecules to the nucleic acid molecules, thereby producing a first spike-in sample; (b) extracting nucleic acids from the first spike-in sample; (c) processing at least a subset of the extracted nucleic acids, thereby producing a processed sample, wherein the processing comprises partitioning, tagging, and/or amplifying at least a subset of the first spike-in sample; (d) enriching for at least a subset of the processed sample, thereby producing an enriched sample; (e) sequencing at least a subset of the enriched sample to generate sequence reads; (0 analyzing the sequence reads to generate fragment size scores of the subset of fragment size control molecules; and (g) comparing the fragment size scores with fragment size thresholds.

### CROSS REFERENCE

This application claims the benefit of, and priority to, U.S Provisional Application No. 62/783,046, filed on Dec. 20, 2018, which application is incorporated herein by reference in its entirety.

### BACKGROUND

Current methods of cancer diagnostic assays of cell-free nucleic acids (e.g., cell-free DNA or cell-free RNA) focus on the detection of tumor-related somatic variants, including single nucleotide variants (SNVs), copy number variations (CNVs), fusions, and indels (i.e., insertions or deletions), which are all mainstream targets for liquid biopsy. There is growing evidence that the size distribution and fragmentation pattern in cell-free DNA can provide information on the source of cell-free DNA and disease level. The size distribution and the fragmentation pattern of the cell-free DNA, when combined with somatic mutation calling, can yield a more comprehensive assessment of tumor status than that available from either approach alone.

### SUMMARY

The present disclosure provides methods, compositions, and systems for analyzing nucleic acids using fragment size molecules.

In one aspect, the present disclosure provides a method for analyzing of nucleic acid molecules in a sample of polynucleotides, comprising: a) adding a subset of fragment size control molecules to the nucleic acid molecules in the sample of cell-free polynucleotides, thereby producing a first spike-in sample; b) extracting nucleic acids from the first spike-in sample; c) processing at least a subset of the extracted nucleic acids, thereby producing a processed sample, wherein the processing comprises partitioning, tagging, and/or amplifying at least a subset of the first spike-in sample; d) enriching for at least a subset of the processed sample, thereby producing an enriched sample; e) sequencing at least a subset of the enriched sample to generate a plurality of sequence reads; and f) analyzing the plurality of sequence reads to generate a plurality of fragment size scores of the subset of fragment size control molecules. In some embodiments, the method further comprises prior to c), adding a second subset of fragment size control molecules, thereby producing a second spike-in sample. In some embodiments, the method further comprises prior to d) adding a third subset of fragment size control molecules, thereby producing a third spike-in sample. In some embodiments, the method comprises prior to e) adding a fourth subset of fragment size control molecules, thereby producing a fourth spike-in sample.

In some embodiments, the sample of polynucleotides is a sample of cell-free polynucleotides. In some embodiments, the sample of polynucleotides is selected from the group consisting of a sample of cell-free DNA, and a sample of cell-free RNA. In some embodiments, the sample of cell-free polynucleotides is a cell-free DNA. In some embodiments, the cell-free DNA is between 1 ng and 500 ng. In some embodiments, the concentration of the fragment size control molecules is between 1 attomole and 10 picomoles

In another aspect, the present disclosure provides a set of fragment size control molecules, comprising at least one subset of pre-determined fragment size control molecules, wherein the at least one subset of pre-determined fragment size control molecules comprises a plurality of fragment size control molecules comprising a fragment size region. In some embodiments, the fragment size control molecules further comprise an identifier region

In some embodiments, the at least one subset comprises at least one group of fragment size control molecules.

In some embodiments, the subset of fragment size control molecules comprises a plurality of fragment size control molecules comprising a fragment size region. In some embodiments, the fragment size control molecules further comprises an identifier region. In some embodiments, the subsets comprises at least one group of fragment size control molecules.

In some embodiments, the fragment size regions of the fragment size control molecules in a group are of same length. In some embodiments, the length of the fragment size region in a first group of fragment size control molecules is different from the length of the fragment size region in a second group of fragment size control molecules.

In some embodiments, the identifier region is on one or both sides of the fragment size region. In some embodiment, the identifier region comprises a molecular barcode.

In some embodiments, the plurality of fragment control molecules comprises one or more primer binding sites. In some embodiments, the one or more primer binding sites are in the identifier region.

In some embodiments, the fragment size regions of the fragment size control molecules in a group comprise the same oligonucleotide sequence. In some embodiments, the fragment size regions of the fragment size control molecules in a group comprise at least two distinguishable oligonucleotide sequences. In some embodiment, the fragment size regions of the fragment size control molecules in a first subset of pre-determined fragment size control molecules comprises an oligonucleotide sequence distinguishable from the oligonucleotide sequence of the fragment size regions of the fragment size control molecules in a second subset of pre-determined fragment size control molecules.

In some embodiments, the length of the fragment size region is at least 10 bp, at least 50 bp, at least 60 bp, at least 70 bp, at least 80 bp, at least 90 bp, at least 100 bp, at least 120 bp, at least 150 bp, at least 200 bp, at least 250 bp, at least 300 bp, at least 400 bp, at least 500 bp, at least 600 bp, at least 700 bp, at least 800 bp, at least 900 bp, or at least 1000 bp. In some embodiments, the length of the fragment size region is between 10 bp and 1000 bp.

In some embodiments, each subset of the at least one subset ofpre-determined fragment size control molecules is in equimolar concentration. In some embodiments, each subset of the at least one subset of pre-determined fragment size control molecules is in non-equimolar concentration. In some embodiments, each group of the at least one group of fragment size control molecules in the at least one subset is in equimolar concentration. In some embodiments, each group of the at least one group of fragment size control molecules in the at least one subset is in non-equimolar concentration.

In some embodiments, each of the subsets of the fragment size control molecules is in equimolar concentration. In some embodiments, each of the subsets of the fragment size control molecules is in non-equimolar concentration. In some embodiments, each of the groups of the fragment size control molecules in the subset is in equimolar concentration. In some embodiments, each of the groups of the fragment size control molecules in the subset is in non-equimolar concentration.

In another aspect, the present disclosure provides a method for evaluating the fragment size bias in the analysis of nucleic acid molecules in a sample of cell-free polynucleotides, comprising: a) adding a first subset of fragment size control molecules to the nucleic acid molecules in the sample of polynucleotides, thereby producing a first spike-in sample; b) extracting nucleic acids from the first spike-in sample; c) adding a second subset of fragment size control molecules to the extracted nucleic acids, thereby producing a second spike-in sample; d) processing at least a subset of the second spike-in sample, thereby producing a processed sample, wherein the processing comprises partitioning, tagging, and/or amplifying the at least the subset of the second spike-in sample; e) adding a third subset of fragment size control molecules to the processed sample, thereby producing a third spike-in sample; f) enriching for at least a subset of the third spike-in sample, thereby producing an enriched sample; g) adding a fourth subset of fragment size control molecules to at least a subset of the enriched sample, thereby producing a fourth spike-in sample; h) sequencing the fourth spike-in sample to generate a plurality of sequence reads; and i) analyzing the plurality of sequence reads to generate a plurality of fragment size scores of the first subset of fragment size control molecules, the second subset of fragment size control molecules, the third subset of fragment size control molecules, and/or the fourth subset of fragment size control molecules. In some embodiments, the method further comprises comparing the plurality of fragment size scores with a plurality of fragment size thresholds. In some embodiments, the method further comprises optimizing the analysis of nucleic acid molecules in the sample of polynucleotides based on the plurality of fragment size scores. In some embodiments the method further comprises correcting for fragment size bias in the analysis of nucleic acid molecules in the sample of cell-free polynucleotides using the plurality of fragment size scores. In some embodiments, the method further comprises classifying the method as (i) being a success, if at least one of the plurality of fragment size scores is within a corresponding fragment size threshold of the plurality of fragment size thresholds; or (ii) being unsuccessful, if at least one of the plurality of fragment size scores is not within the corresponding fragment size threshold of the plurality of fragment size thresholds.

In another aspect, the present disclosure provides method of detecting contamination of a first sample with a second sample, comprising, for each of the first sample and the second sample: (a) adding a subset of fragment size control molecules to generate a first spiked-in sample, wherein the subset of fragment size control molecules added to the first sample can be distinguished from the subset of fragment size control molecules added to the second sample; (b) extracting nucleic acids from the first spike-in; (c) processing at least a subset of the extracted nucleic acids, thereby producing a processed sample, wherein the processing comprises partitioning, tagging and/or amplifying at least a subset of the first spike-in sample; (d) enriching for at least a subset of the processed sample; (e) sequencing at least a subset of the enriched sample to generate a plurality of sequence reads; and (f) analyzing the plurality of sequence reads to generate one or more contamination scores of the subset of fragment size control molecules. In some embodiments, the method further comprises, prior to c), adding a second subset of fragment size control molecules, thereby producing a second spike-in sample, wherein the subset of fragment size control molecules added to the first sample can be distinguished from the subset of fragment size control molecules added to the second sample. In some embodiments, the method further comprises, prior to d), adding a third subset of fragment size control molecules, thereby producing a third spike-in sample, wherein the subset of fragment size control molecules added to the first sample can be distinguished from the subset of fragment size control molecules added to the second sample. In some embodiments, the method further comprises, prior to e), adding a fourth subset of fragment size control molecules, thereby producing a fourth spike-in sample., wherein the subset of fragment size control molecules added to the first sample can be distinguished from the subset of fragment size control molecules added to the second sample.

In another aspect, the present disclosure provides a method of detecting contamination of a first sample with a second sample, comprising, for each of the first sample and the second sample: (a) adding a first subset of fragment size control molecules to generate a first spiked-in sample, wherein the first subset of fragment size control molecules added to the first sample can be distinguished from the first subset of fragment size control molecules added to the second sample; (b) extracting nucleic acids from the first spike-in; (c) adding a second subset of fragment size control molecules to the extracted nucleic acids, thereby producing a second spike-in sample, wherein the second subset of fragment size control molecules added to the first sample can be distinguished from the second subset of fragment size control molecules added to the second sample; (d) processing at least a subset of the extracted nucleic acids, thereby producing a processed sample, wherein the processing comprises partitioning, tagging and/or amplifying at least a subset of the first spike-in sample; (e) adding a third subset of fragment size control molecules to the extracted nucleic acids, thereby producing a third spike-in sample, wherein the third subset of fragment size control molecules added to the first sample can be distinguished from the third subset of fragment size control molecules added to the second sample; (f) enriching for at least a subset of the processed sample; (g) adding a fourth subset of fragment size control molecules to the extracted nucleic acids, thereby producing a fourth spike-in sample, wherein the fourth subset of fragment size control molecules added to the first sample can be distinguished from the fourth subset of fragment size control molecules added to the second sample; (h) sequencing at least a subset of the enriched sample to generate a plurality of sequence reads; and (i) analyzing the plurality of sequence reads to generate one or more contamination scores of the subset of fragment size control molecules.

In some embodiments, the method further comprises comparing at least one or more contamination scores with at least one or more contamination thresholds. In some embodiments, the method further comprises classifying the first sample as (i) being contaminated with the second sample, if at least one or more of the contamination scores is not within a corresponding contamination threshold of the one or more contamination thresholds; or (ii) being not contaminated with the second sample, if at least one or more of the contamination scores is within the corresponding contamination threshold of the one or more contamination thresholds.

In some embodiments, the partitioning comprises partitioning the nucleic acid molecules of the at least the subset of the second spike-in sample into a plurality of partitioned sets. In some embodiments, the plurality of partitioned sets comprises nucleic acid molecules of the second spike-in sample partitioned based on the epigenetic modification level of the nucleic acid molecules of the second spike-in sample.

In some embodiments, the tagging comprises attaching a set of tags to the nucleic acids to produce a population of tagged nucleic acids, wherein the tagged nucleic acids comprise one or more tags. In some embodiments, the set of tags used in a first partitioned set of a plurality of partitioned sets resulting from the partitioning is different from the set of tags used in a second partitioned set of the plurality of partitioned sets. In some embodiments, the set of tags is attached to the nucleic acids by ligation of adapters to the nucleic acids, wherein the adapters comprise one or more tags.

In some embodiments, the sample of polynucleotides is a sample of cell-free polynucleotides. In some embodiments, the sample of polynucleotides is selected from the group consisting of a sample of cell-free DNA, and a sample of cell-free RNA. In some embodiments, the sample of cell-free polynucleotides is a cell-free DNA. In some embodiments, the cell-free DNA is between 1 ng and 500 ng. In some embodiments, the concentration of the fragment size control molecules is between 1 attomole and 10 picomoles

In another aspect, the present disclosure provides a set of fragment size control molecules, comprising at least one subset of pre-determined fragment size control molecules, wherein the at least one subset of pre-determined fragment size control molecules comprises a plurality of fragment size control molecules comprising a fragment size region. In some embodiments, the fragment size control molecules further comprise an identifier region

In some embodiments, the at least one subset comprises at least one group of fragment size control molecules.

In some embodiments, the subset of fragment size control molecules comprises a plurality of fragment size control molecules comprising a fragment size region. In some embodiments, the fragment size control molecules further comprises an identifier region. In some embodiments, the subsets comprises at least one group of fragment size control molecules.

In some embodiments, the fragment size regions of the fragment size control molecules in a group are of same length. In some embodiments, the length of the fragment size region in a first group of fragment size control molecules is different from the length of the fragment size region in a second group of fragment size control molecules.

In some embodiments, the identifier region is on one or both sides of the fragment size region. In some embodiment, the identifier region comprises a molecular barcode.

In some embodiments, the plurality of fragment control molecules comprises one or more primer binding sites. In some embodiments, the one or more primer binding sites are in the identifier region.

In some embodiments, the fragment size regions of the fragment size control molecules in a group comprise the same oligonucleotide sequence. In some embodiments, the fragment size regions of the fragment size control molecules in a group comprise at least two distinguishable oligonucleotide sequences. In some embodiment, the fragment size regions of the fragment size control molecules in a first subset of pre-determined fragment size control molecules comprises an oligonucleotide sequence distinguishable from the oligonucleotide sequence of the fragment size regions of the fragment size control molecules in a second subset of pre-determined fragment size control molecules.

In some embodiments, the length of the fragment size region is at least 10 bp, at least 50 bp, at least 60 bp, at least 70 bp, at least 80 bp, at least 90 bp, at least 100 bp, at least 120 bp, at least 150 bp, at least 200 bp, at least 250 bp, at least 300 bp, at least 400 bp, at least 500 bp, at least 600 bp, at least 700 bp, at least 800 bp, at least 900 bp, or at least 1000 bp. In some embodiments, the length of the fragment size region is between 10 bp and 1000 bp.

In some embodiments, each subset of the at least one subset ofpre-determined fragment size control molecules is in equimolar concentration. In some embodiments, each subset of the at least one subset of pre-determined fragment size control molecules is in non-equimolar concentration. In some embodiments, each group of the at least one group of fragment size control molecules in the at least one subset is in equimolar concentration. In some embodiments, each group of the at least one group of fragment size control molecules in the at least one subset is in non-equimolar concentration.

In some embodiments, each of the subsets of the fragment size control molecules is in equimolar concentration. In some embodiments, each of the subsets of the fragment size control molecules is in non-equimolar concentration. In some embodiments, each of the groups of the fragment size control molecules in the subset is in equimolar concentration. In some embodiments, each of the groups of the fragment size control molecules in the subset is in non-equimolar concentration.

In another aspect, the present disclosure provides a method for producing a sequencing library of a sample of cell-free polynucleotides, comprising: a) adding a subset of fragment size control molecules to the sample, thereby producing a first spike-in sample; b) extracting nucleic acids from the first spike-in sample; c) processing at least a subset of the extracted nucleic acids, thereby producing a processed sample, wherein the processing comprises partitioning, tagging, and/or amplifying at least a subset of the first spike-in sample; and d) enriching for at least a subset of the processed sample. In some embodiments, the method further comprises, prior to c), adding a second subset of fragment size control molecules, thereby producing a second spike-in sample. In some embodiments, the method further comprises prior to d), adding a third subset of fragment size control molecules, thereby producing a third spike-in sample. In some embodiments, the method further comprises e) adding a fourth subset of fragment size control molecules, thereby producing a fourth spike-in sample.

In some embodiments, the at least one subset comprises at least one group of fragment size control molecules.

In some embodiments, the subset of fragment size control molecules comprises a plurality of fragment size control molecules comprising a fragment size region. In some embodiments, the fragment size control molecules further comprises an identifier region. In some embodiments, the subsets comprises at least one group of fragment size control molecules.

In some embodiments, the fragment size regions of the fragment size control molecules in a group are of same length. In some embodiments, the length of the fragment size region in a first group of fragment size control molecules is different from the length of the fragment size region in a second group of fragment size control molecules.

In some embodiments, the identifier region is on one or both sides of the fragment size region. In some embodiment, the identifier region comprises a molecular barcode.

In some embodiments, the plurality of fragment control molecules comprises one or more primer binding sites. In some embodiments, the one or more primer binding sites are in the identifier region.

In some embodiments, the fragment size regions of the fragment size control molecules in a group comprise the same oligonucleotide sequence. In some embodiments, the fragment size regions of the fragment size control molecules in a group comprise at least two distinguishable oligonucleotide sequences. In some embodiment, the fragment size regions of the fragment size control molecules in a first subset of pre-determined fragment size control molecules comprises an oligonucleotide sequence distinguishable from the oligonucleotide sequence of the fragment size regions of the fragment size control molecules in a second subset of pre-determined fragment size control molecules.

In some embodiments, the length of the fragment size region is at least 10 bp, at least 50 bp, at least 60 bp, at least 70 bp, at least 80 bp, at least 90 bp, at least 100 bp, at least 120 bp, at least 150 bp, at least 200 bp, at least 250 bp, at least 300 bp, at least 400 bp, or at least 500 bp. In some embodiments, the length of the fragment size region is between 10 bp and 1000 bp.

In some embodiments, each subset of the at least one subset ofpre-determined fragment size control molecules is in equimolar concentration. In some embodiments, each subset of the at least one subset of pre-determined fragment size control molecules is in non-equimolar concentration. In some embodiments, each group of the at least one group of fragment size control molecules in the at least one subset is in equimolar concentration. In some embodiments, each group of the at least one group of fragment size control molecules in the at least one subset is in non-equimolar concentration.

In yet another aspect, the present disclosure provide a population of nucleic acids, comprising: (a) a set of fragment size control molecules, comprising at least one subset of pre-determined fragment size control molecules, wherein the at least one subset of pre-determined fragment size control molecules comprises a plurality of fragment size control molecules comprising a fragment size region; and (b) a set of nucleic acid molecules in a sample of polynucleotides from a subject. In some embodiment, the at least one subset comprises at least one group of fragment size control molecules. In some embodiments, the fragment size regions of the fragment size control molecules in a group are of same length. In some embodiments, the length of the fragment size region in a first group of fragment size control molecules is different from the length of the fragment size region in a second group of fragment size control molecules. In some embodiments, the fragment size control molecules further comprise an identifier region. In some embodiments, the identifier region is on one or both sides of the fragment size region. In some embodiments, the identifier region comprises a molecular barcode. In some embodiments, the plurality of fragment control molecules comprises one or more primer binding sites. In some embodiments, the one or more primer binding sites are in the identifier region. In some embodiments, the fragment size regions of the fragment size control molecules in a group comprise the same oligonucleotide sequence. In some embodiments, the fragment size regions of the fragment size control molecules in a group comprise at least two distinguishable oligonucleotide sequences. In some embodiments, the fragment size regions of the fragment size control molecules in a first subset of pre-determined fragment size control molecules comprises an oligonucleotide sequence distinguishable from the oligonucleotide sequence of the fragment size regions of the fragment size control molecules in a second subset of pre-determined fragment size control molecules. In some embodiments, the length of the fragment size region is at least 10 bp, at least 50 bp, at least 60 bp, at least 70 bp, at least 80 bp, at least 90 bp, at least 100 bp, at least 120 bp, at least 150 bp, at least 200 bp, at least 250 bp, at least 300 bp, at least 400 bp, at least 500 bp, at least 600 bp, at least 700 bp, at least 800 bp, at least 900 bp, or at least 1000 bp. In some embodiments, the length of the fragment size region is between 10 bp and 1000 bp. In some embodiments, each subset of the at least one subset of pre-determined fragment size control molecules is in equimolar concentration. In some embodiments, each group of the at least one group of fragment size control molecules in the at least one subset is in equimolar concentration. In some embodiments, each group of the at least one group of fragment size control molecules in the at least one subset is in non-equimolar concentration.

In another aspect, the present disclosure provides a system comprising a controller comprising or capable of accessing, computer readable media comprising non-transitory computer-executable instructions which, when executed by at least one electronic processor perform a method comprising: a) adding a subset of fragment size control molecules to the nucleic acid molecules in the sample of polynucleotides, thereby producing a first spike-in sample; b) extracting nucleic acids from the first spike-in sample; c) processing at least a subset of the extracted nucleic acids, thereby producing a processed sample, wherein the processing comprises partitioning, tagging, and/or amplifying at least a subset of the first spike-in sample; d) enriching for at least a subset of the processed sample, thereby producing an enriched sample; e) sequencing at least a subset of the enriched sample to generate a plurality of sequence reads; and f) analyzing the plurality of sequence reads to generate a plurality of fragment size scores of the subset of fragment size control molecules. In some embodiments, the method further comprises prior to c), adding a second subset of fragment size control molecules, thereby producing a second spike-in sample. In some embodiments, the method further comprises prior to d), adding a third subset of fragment size control molecules, thereby producing a third spike-in sample. In some embodiments, the method further comprises prior to e), adding a fourth subset of fragment size control molecules, thereby producing a fourth spike-in sample.

In some embodiments, the sample of polynucleotides is a sample of cell-free polynucleotides. In some embodiments, the sample of polynucleotides is selected from the group consisting of a sample of cell-free DNA, and a sample of cell-free RNA. In some embodiments, the sample of cell-free polynucleotides is a cell-free DNA. In some embodiments, the cell-free DNA is between 1 ng and 500 ng. In some embodiments, the concentration of the fragment size control molecules is between 1 attomole and 10 picomoles

In another aspect, the present disclosure provides a set of fragment size control molecules, comprising at least one subset of pre-determined fragment size control molecules, wherein the at least one subset of pre-determined fragment size control molecules comprises a plurality of fragment size control molecules comprising a fragment size region. In some embodiments, the fragment size control molecules further comprise an identifier region

In some embodiments, the at least one subset comprises at least one group of fragment size control molecules.

In some embodiments, the subset of fragment size control molecules comprises a plurality of fragment size control molecules comprising a fragment size region. In some embodiments, the fragment size control molecules further comprises an identifier region. In some embodiments, the subsets comprises at least one group of fragment size control molecules.

In some embodiments, the fragment size regions of the fragment size control molecules in a group are of same length. In some embodiments, the length of the fragment size region in a first group of fragment size control molecules is different from the length of the fragment size region in a second group of fragment size control molecules.

In some embodiments, the identifier region is on one or both sides of the fragment size region. In some embodiment, the identifier region comprises a molecular barcode.

In some embodiments, the plurality of fragment control molecules comprises one or more primer binding sites. In some embodiments, the one or more primer binding sites are in the identifier region.

In some embodiments, the fragment size regions of the fragment size control molecules in a group comprise the same oligonucleotide sequence. In some embodiments, the fragment size regions of the fragment size control molecules in a group comprise at least two distinguishable oligonucleotide sequences. In some embodiment, the fragment size regions of the fragment size control molecules in a first subset of pre-determined fragment size control molecules comprises an oligonucleotide sequence distinguishable from the oligonucleotide sequence of the fragment size regions of the fragment size control molecules in a second subset of pre-determined fragment size control molecules.

In some embodiments, the length of the fragment size region is at least 10 bp, at least 50 bp, at least 60 bp, at least 70 bp, at least 80 bp, at least 90 bp, at least 100 bp, at least 120 bp, at least 150 bp, at least 200 bp, at least 250 bp, at least 300 bp, at least 400 bp, at least 500 bp, at least 600 bp, at least 700 bp, at least 800 bp, at least 900 bp, or at least 1000 bp. In some embodiments, the length of the fragment size region is between 10 bp and 1000 bp.

In some embodiments, each subset of the at least one subset ofpre-determined fragment size control molecules is in equimolar concentration. In some embodiments, each subset of the at least one subset of pre-determined fragment size control molecules is in non-equimolar concentration. In some embodiments, each group of the at least one group of fragment size control molecules in the at least one subset is in equimolar concentration. In some embodiments, each group of the at least one group of fragment size control molecules in the at least one subset is in non-equimolar concentration.

In some embodiments, each of the subsets of the fragment size control molecules is in equimolar concentration. In some embodiments, each of the subsets of the fragment size control molecules is in non-equimolar concentration. In some embodiments, each of the groups of the fragment size control molecules in the subset is in equimolar concentration. In some embodiments, each of the groups of the fragment size control molecules in the subset is in non-equimolar concentration.

In another aspect, the present disclosure provides a system comprising a controller comprising or capable of accessing, computer readable media comprising non-transitory computer-executable instructions which, when executed by at least one electronic processor perform a method comprising: a) adding a first subset of fragment size control molecules to the nucleic acid molecules in the sample of polynucleotides, thereby producing a first spike-in sample b) extracting nucleic acids from the first spike-in sample; c) adding a second subset of fragment size control molecules to the extracted nucleic acids, thereby producing a second spike-in sample; d) processing at least a subset of the second spike-in sample, thereby producing a processed sample wherein the processing comprises partitioning, tagging, and/or amplifying the at least the subset of the second spike-in sample; e) adding a third subset of fragment size control molecules to the processed sample, thereby producing a third spike-in sample; f) enriching for at least a subset of the third spike-in sample, thereby producing an enriched sample; g) adding a fourth subset of fragment size control molecules to at least a subset of the enriched sample, thereby producing a fourth spike-in sample; h) sequencing the fourth spike-in sample to generate a plurality of sequence reads; and i) analyzing the plurality of sequence reads to generate a plurality of fragment size scores of the first subset of fragment size control molecules, the second subset of fragment size control molecules, the third subset of fragment size control molecules, and/or the fourth subset of fragment size control molecules. In some embodiments, the method further comprises comparing the plurality of fragment size scores with a plurality of fragment size thresholds. In some embodiments, the method further comprises optimizing the analysis of nucleic acid molecules in the sample of polynucleotides based on the plurality of fragment size scores. In some embodiments, the method further comprises correcting for fragment size bias in the analysis of nucleic acid molecules in the sample of polynucleotides using the plurality of fragment size scores. In some embodiments, the method further comprises classifying the method as (i) being a success, if each of the plurality of fragment size scores is within a corresponding fragment size threshold of the plurality of fragment size thresholds; or (ii) being unsuccessful, if at least one of the plurality of fragment size scores is not within the corresponding fragment size threshold of the plurality of fragment size thresholds. In some embodiments, the method further comprises comparing at least one of the plurality of fragment size scores with at least one of plurality of contamination thresholds. In some embodiments, the method further comprises classifying the sample as (i) being contaminated with another sample, if at least one of the plurality of fragment size scores is not within a corresponding contamination threshold of the plurality of contamination thresholds; or (ii) being not contaminated with another sample, if at least one of the plurality of fragment size scores is within the corresponding contamination threshold of the plurality of contamination thresholds.

In some embodiments, the sample of polynucleotides is a sample of cell-free polynucleotides. In some embodiments, the sample of polynucleotides is selected from the group consisting of a sample of cell-free DNA, and a sample of cell-free RNA. In some embodiments, the sample of cell-free polynucleotides is a cell-free DNA. In some embodiments, the cell-free DNA is between 1 ng and 500 ng. In some embodiments, the concentration of the fragment size control molecules is between 1 attomole and 10 picomoles

In another aspect, the present disclosure provides a set of fragment size control molecules, comprising at least one subset of pre-determined fragment size control molecules, wherein the at least one subset of pre-determined fragment size control molecules comprises a plurality of fragment size control molecules comprising a fragment size region. In some embodiments, the fragment size control molecules further comprise an identifier region

In some embodiments, the at least one subset comprises at least one group of fragment size control molecules.

In some embodiments, the subset of fragment size control molecules comprises a plurality of fragment size control molecules comprising a fragment size region. In some embodiments, the fragment size control molecules further comprises an identifier region. In some embodiments, the subsets comprises at least one group of fragment size control molecules.

In some embodiments, the fragment size regions of the fragment size control molecules in a group are of same length. In some embodiments, the length of the fragment size region in a first group of fragment size control molecules is different from the length of the fragment size region in a second group of fragment size control molecules.

In some embodiments, the identifier region is on one or both sides of the fragment size region. In some embodiment, the identifier region comprises a molecular barcode.

In some embodiments, the plurality of fragment control molecules comprises one or more primer binding sites. In some embodiments, the one or more primer binding sites are in the identifier region.

In some embodiments, the fragment size regions of the fragment size control molecules in a group comprise the same oligonucleotide sequence. In some embodiments, the fragment size regions of the fragment size control molecules in a group comprise at least two distinguishable oligonucleotide sequences. In some embodiment, the fragment size regions of the fragment size control molecules in a first subset of pre-determined fragment size control molecules comprises an oligonucleotide sequence distinguishable from the oligonucleotide sequence of the fragment size regions of the fragment size control molecules in a second subset of pre-determined fragment size control molecules.

In some embodiments, the length of the fragment size region is at least 10 bp, at least 50 bp, at least 60 bp, at least 70 bp, at least 80 bp, at least 90 bp, at least 100 bp, at least 120 bp, at least 150 bp, at least 200 bp, at least 250 bp, at least 300 bp, at least 400 bp, at least 500 bp, at least 600 bp, at least 700 bp, at least 800 bp, at least 900 bp, or at least 1000 bp. In some embodiments, the length of the fragment size region is between 10 bp and 1000 bp.

In some embodiments, each subset of the at least one subset ofpre-determined fragment size control molecules is in equimolar concentration. In some embodiments, each subset of the at least one subset of pre-determined fragment size control molecules is in non-equimolar concentration. In some embodiments, each group of the at least one group of fragment size control molecules in the at least one subset is in equimolar concentration. In some embodiments, each group of the at least one group of fragment size control molecules in the at least one subset is in non-equimolar concentration.

In some embodiments, each of the subsets of the fragment size control molecules is in equimolar concentration. In some embodiments, each of the subsets of the fragment size control molecules is in non-equimolar concentration. In some embodiments, each ofthe groups of the fragment size control molecules in the subset is in equimolar concentration. In some embodiments, each of the groups of the fragment size control molecules in the subset is in non-equimolar concentration.

In another aspect, the present disclosure provides a system, comprising a controller comprising or capable of accessing, computer readable media comprising non-transitory computer-executable instructions which, when executed by at least one electronic processor perform a method comprising: a) adding a subset of fragment size control molecules to the sample, thereby producing a first spike-in sample; b) extracting nucleic acids from the first spike-in sample; c) processing at least a subset of the extracted nucleic acids, thereby producing a processed sample, wherein the processing comprises partitioning, tagging, and/or amplifying at least a subset of the first spike-in sample; and d) enriching for at least a subset of the processed sample. In some embodiments, the method further comprise prior to c), adding a second subset of fragment size control molecules, thereby producing a second spike-in sample. In some embodiments, the method further comprises prior to d), adding a third subset of fragment size control molecules, thereby producing a third spike-in sample. In some embodiments, the method further comprises e) adding a fourth subset of fragment size control molecules, thereby producing a fourth spike-in sample.

In some embodiments, the sample of polynucleotides is a sample of cell-free polynucleotides. In some embodiments, the sample of polynucleotides is selected from the group consisting of a sample of cell-free DNA, and a sample of cell-free RNA. In some embodiments, the sample of cell-free polynucleotides is a cell-free DNA. In some embodiments, the cell-free DNA is between 1 ng and 500 ng. In some embodiments, the concentration of the fragment size control molecules is between 1 attomole and 10 picomoles

In another aspect, the present disclosure provides a set of fragment size control molecules, comprising at least one subset of pre-determined fragment size control molecules, wherein the at least one subset of pre-determined fragment size control molecules comprises a plurality of fragment size control molecules comprising a fragment size region. In some embodiments, the fragment size control molecules further comprise an identifier region

In some embodiments, the at least one subset comprises at least one group of fragment size control molecules.

In some embodiments, the subset of fragment size control molecules comprises a plurality of fragment size control molecules comprising a fragment size region. In some embodiments, the fragment size control molecules further comprises an identifier region. In some embodiments, the subsets comprises at least one group of fragment size control molecules.

In some embodiments, the fragment size regions of the fragment size control molecules in a group are of same length. In some embodiments, the length of the fragment size region in a first group of fragment size control molecules is different from the length of the fragment size region in a second group of fragment size control molecules.

In some embodiments, the identifier region is on one or both sides of the fragment size region. In some embodiment, the identifier region comprises a molecular barcode.

In some embodiments, the plurality of fragment control molecules comprises one or more primer binding sites. In some embodiments, the one or more primer binding sites are in the identifier region.

In some embodiments, the fragment size regions of the fragment size control molecules in a group comprise the same oligonucleotide sequence. In some embodiments, the fragment size regions of the fragment size control molecules in a group comprise at least two distinguishable oligonucleotide sequences. In some embodiment, the fragment size regions of the fragment size control molecules in a first subset of pre-determined fragment size control molecules comprises an oligonucleotide sequence distinguishable from the oligonucleotide sequence of the fragment size regions of the fragment size control molecules in a second subset of pre-determined fragment size control molecules.

In some embodiments, the length of the fragment size region is at least 10 bp, at least 50 bp, at least 60 bp, at least 70 bp, at least 80 bp, at least 90 bp, at least 100 bp, at least 120 bp, at least 150 bp, at least 200 bp, at least 250 bp, at least 300 bp, at least 400 bp, at least 500 bp, at least 600 bp, at least 700 bp, at least 800 bp, at least 900 bp, or at least 1000 bp. In some embodiments, the length of the fragment size region is between 10 bp and 1000 bp.

In some embodiments, the fragment size control molecules are synthetic molecules. In some embodiments, the fragment size control molecules are generated as amplicons by PCR amplification.

In some embodiments, each subset of the at least one subset ofpre-determined fragment size control molecules is in equimolar concentration. In some embodiments, each subset of the at least one subset of pre-determined fragment size control molecules is in non-equimolar concentration. In some embodiments, each group of the at least one group of fragment size control molecules in the at least one subset is in equimolar concentration. In some embodiments, each group of the at least one group of fragment size control molecules in the at least one subset is in non-equimolar concentration.

In some embodiments, each of the subsets of the fragment size control molecules is in equimolar concentration. In some embodiments, each of the subsets of the fragment size control molecules is in non-equimolar concentration. In some embodiments, each of the groups of the fragment size control molecules in the subset is in equimolar concentration. In some embodiments, each of the groups of the fragment size control molecules in the subset is in non-equimolar concentration.

The disclosure also provides kits for practicing any of the above methods. An exemplary kit comprises: (a) a set of fragment size control molecules, comprising at least one subset of pre-determined fragment size control molecules, wherein the at least one subset of pre-determined fragment size control molecules comprises a plurality of fragment size control molecules comprising a fragment size region.

In some embodiments, the method or system further comprises generating a report which optionally includes information on, and/or information derived from, the analysis of the nucleic acid molecules.

In some embodiments, the results of the systems and/or methods disclosed herein are used as an input to generate a report. The report may be in a paper or electronic format. For example, information on, and/or information derived from, the analysis of nucleic acid molecules, as determined by the methods or systems disclosed herein, can be displayed in such a report. The methods or systems disclosed herein may further comprise a step of communicating the report to a third party, such as the subject from whom the sample derived or a health care practitioner.

The various steps of the methods disclosed herein, or the steps carried out by the systems disclosed herein, may be carried out at the same time or different times, and/or in the same geographical location or different geographical locations, e.g. countries. The various steps of the methods disclosed herein can be performed by the same person or different people.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The accompanying drawings, which are incorporated in and constitute a part of this specification, illustrate certain embodiments, and together with the written description, serve to explain certain principles of the methods, computer readable media, and systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1A is a schematic representation of a method for analyzing nucleic acid molecules in a sample of polynucleotides according to an embodiment of the disclosure. FIG. 1B is a flow chart representation of a method for analyzing nucleic acid molecules in a sample of polynucleotides according to the embodiment shown in FIG. 1A.

FIG. 2A is a schematic representation of a method for analyzing nucleic acid molecules in a sample of polynucleotides according to an embodiment of the disclosure. FIG. 2B is a flow chart representation of a method for analyzing nucleic acid molecules in a sample of polynucleotides according to the embodiment shown in FIG. 2A.

FIG. 3 is a schematic representation of fragment size control molecules suitable for use with some embodiments of the disclosure.

FIG. 4 is a schematic representation of fragment size control molecules suitable for use with some embodiments of the disclosure.

FIG. 5 is a schematic diagram of an example of a system suitable for use with some embodiments of the disclosure.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent upon reading this disclosure and so forth.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Adapter: As used herein, “adapter” refers to a short nucleic acid (e.g., less than about 500 nucleotides, less than about 100 nucleotides, or less than about 50 nucleotides in length) that is typically at least partially double-stranded and the adapters can be attached to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next-generation sequencing (NGS) applications. Adapters can also include binding sites for capture probes, such as an oligonucleotide attached to a flow cell support or the like. Adapters can also include a nucleic acid tag as described herein. Nucleic acid tags are typically positioned relative to amplification primer and sequencing primer binding sites, such that a nucleic acid tag is included in amplicons and sequence reads of a given nucleic acid molecule. Adapters of the same or different sequences can be linked to the respective ends of a nucleic acid molecule. In some embodiments, adapters of the same sequence is linked to the respective ends of the nucleic acid molecule except that the nucleic acid tag differs. In some embodiments, the adapter is a Y-shaped adapter in which one end is blunt ended or tailed as described herein, for joining to a nucleic acid molecule, which is also blunt ended or tailed with one or more complementary nucleotides and the other end of the Y-shaped adapter comprises a non-complimentary sequence which does not hybridize to form a double-strand. In still other example embodiments, an adapter is a bell-shaped adapter that includes a blunt or tailed end for joining to a nucleic acid molecule to be analyzed. Other examples of adapters include T-tailed and C-tailed adapters.

Amplify: As used herein, “amplify” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. Amplification includes but is not limited to polymerase chain reaction (PCR).

Cancer Type: As used herein, “cancer type” refers to a type or subtype of cancer defined, e.g., by histopathology. Cancer type can be defined by any conventional criterion, such as on the basis of occurrence in a given tissue (e.g., blood cancers, central nervous system (CNS), brain cancers, lung cancers (small cell and non-small cell), skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers), unknown primary origin and the like, and/or of the same cell lineage (e.g., carcinoma, sarcoma, lymphoma, cholangiocarcinoma, leukemia, mesothelioma, melanoma, or glioblastoma) and/or cancers exhibiting cancer markers, such as, but not limited to, Her2, CA15-3, CA19-9, CA-125, CEA, AFP, PSA, HCG, hormone receptor and NMP-22. Cancers can also be classified by stage (e.g., stage 1, 2, 3, or 4) and whether of primary or secondary origin.

Cell-Free Nucleic Acid: As used herein, “cell-free nucleic acid” refers to nucleic acids not contained within or otherwise bound to a cell or, in some embodiments, nucleic acids remaining in a sample following the removal of intact cells. Cell-free nucleic acids can include, for example, all non-encapsulated nucleic acids sourced from a bodily fluid (e.g., blood, plasma, serum, urine, cerebrospinal fluid (CSF), etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell death processes, e.g., cellular necrosis, apoptosis, or the like. Some cell-free nucleic acids are released into bodily fluid from cancer cells, e.g., circulating tumor DNA (ctDNA). Others are released from healthy cells. CtDNA can be non-encapsulated tumor-derived fragmented DNA. A cell-free nucleic acid can have one or more epigenetic modifications, for example, a cell-free nucleic acid can be acetylated, 5-methylated, and/or hydroxy methylated.

Cellular Nucleic Acids: As used herein, “cellular nucleic acids” means nucleic acids that are disposed within one or more cells from which the nucleic acids have originated, at least at the point a sample is taken or collected from a subject, even if those nucleic acids are subsequently removed (e.g., via cell lysis) as part of a given analytical process.

Contamination: As used herein, the terms “contamination” or “contamination of samples” refer to any chemical or digital contamination of one sample with another sample. Contamination can be due to a variety of sources, such as, but not limited to: physical carryover of liquids between samples (e.g. pipetting, automated liquid handling via sample preparation or sequencer systems, manipulating amplified material); demultiplexing artifacts (e.g. base call errors confounding sample indexes that have limited pairwise Hamming distance; insertion/deletion confounding sample indexes that have limited pairwise edit distance) and reagent impurities (e.g. sample index oligos contaminated (through either carryover of synthesis errors) with oligos containing another sample index).

Contamination score: As used herein, “contamination score” refers to a score in a first sample that represents the presence of fragment size control molecules added to a second sample. In some embodiments, the subset identifier barcode used in one subset in one sample can be different from the subset identifier used in the other subsets of the same sample and the subsets of the other samples. In these embodiments, from the sequence of the subset identifier barcode present in the fragment size control molecules, the presence of fragment size control molecules that belong to a different second sample can be identified. In some embodiments, the contamination score can be specific for each type/group of fragment size control molecules used in a subset (i.e., a separate contamination score for different lengths/groups of fragment size control molecules used in a subset) or it could be an overall score that represents the different lengths/groups of fragments size control molecules. In some embodiments, the contamination score can be estimated based on the number of fragment size control molecules that belong to a different second sample. In some embodiments, the contamination score can be estimated based on the number of sequencing reads of the fragment size control molecules that belong to a different second sample. In some embodiments, the contamination score of a sample can be estimated based on the fraction or percentage of number of sequencing reads of the fragment size control molecules that belong to other samples to the number of sequencing reads of the fragment size control molecules added to that sample. In some embodiments, the contamination score of a sample can be estimated based on the fraction or percentage of number of molecules of the fragment size control molecules that belong to other samples to the number of molecules of the fragment size control molecules added to that sample. In these embodiments, the fragment size control molecules added to that molecule can be identified from the subset identifier barcode of the fragment size control molecules.

Contamination threshold: As used herein, “contamination threshold” refers to a predetermined threshold value or range used to evaluate the contamination of samples in the analysis of nucleic acid molecules in a sample. These thresholds can also be used to optimize the assay in any of the steps such as extraction, library preparation, enrichment, washing/clean up and sequencing. In some embodiments, the contamination threshold can be specific for each type/group of fragment size control molecules used in a subset (i.e., a separate contamination threshold for different lengths/groups of fragment size control molecules used in a subset). In some embodiments, the contamination threshold can be an overall threshold for the different lengths/groups of fragments size control molecules in a subset or an overall threshold for the fragment size control molecules used in one or more subsets added in the assay. In some embodiments, each group in a subset has a particular contamination threshold. For example, a set of fragment size control molecules comprises two subsets—subset 1 and subset 2. If each subset comprises two groups—G11 and G12 (for subset 1) and G21 and G22 (for subset 2), then each of the four groups can have a contamination score (S)—S11 for G11, S12 for G12, S21 for G21, and S22 for G22, based on the recovery of the fragment size control molecules. Each group in a subset can have a separate predetermined contamination threshold. In this example, T11, T12, T21, and T22 are the contamination thresholds for G11, G12, G21, and G22 respectively. The threshold can be in terms of percentage or fraction, and the threshold can be a threshold range instead of a particular threshold value. For the method to be considered a success, at least one of these contamination scores should be within its corresponding contamination threshold. In some embodiments, if each of the plurality of contamination scores is within the corresponding contamination threshold, then the method is classified as performing successfully.

Coverage: As used herein, the terms “coverage”, “total molecule count”, or “total allele count” are used interchangeably. They refer to the total number of DNA molecules at a particular genomic position in a given sample.

Deoxyribonucleic Acid or Ribonucleic Acid: As used herein, “deoxyribonucleic acid” or “DNA” refers to a natural or modified nucleotide which has a hydrogen group at the 2′-position of the sugar moiety. DNA typically includes a chain of nucleotides comprising four types of nucleotide bases; adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA typically includes a chain of nucleotides comprising four types of nucleotide bases; A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “sequence information,” “nucleic acid sequence,” “nucleotide sequence”, “genomic sequence,” “genetic sequence,” or “fragment sequence,” or “nucleic acid sequencing read” denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

DNA sequence: As used herein, “DNA sequence” or “sequence” refers to “raw sequence reads” and/or “consensus sequences.” Raw sequence reads are the output of a DNA sequencer, and typically include redundant sequences of the same parent molecule, for example after amplification. “Consensus sequences” are sequences derived from redundant sequences of a parent molecule intended to represent the sequence of the original parent molecule. Consensus sequences can be produced by voting (wherein each majority nucleotide, e.g., the most commonly observed nucleotide at a given base position, among the sequences is the consensus nucleotide) or other approaches such as comparing to a reference genome. Consensus sequences can be produced by tagging original parent molecules with unique or non-unique molecular tags, which allow tracking of the progeny sequences (e.g., after amplification) by tracking of the tag and/or use of sequence read internal information. Examples of tagging or barcoding, and uses of tags or barcodes, are provided in, for example, U.S. Patent Pub. Nos. 2015/0368708, 2015/0299812, 2016/0040229, and 2016/0046986, each of which is entirely incorporated herein by reference.

Epigenetic modification: As used herein, “epigenetic modification” refers to a modification of the base of the nucleotide(s) in the nucleic acid molecules that affects the regulation and/or gene expression of that particular nucleic acid sequence. The modification can be a chemical modification of the nucleotides' base. In some cases, the modification can be methylation of the nucleotides' base. For example, the modification can be methylation of cytosine, resulting in 5-methylcytosine.

Epigenetic state: As used herein, “epigenetic state” refers to the level/degree of epigenetic modification of the nucleic acid molecules. For example, if the epigenetic modification is DNA methylation (or hydroxy methylation), then the epigenetic state can refer to the presence or absence of methylation on a DNA base (e.g., cytosine) or to the degree of methylation in a nucleic acid sequence (e.g., highly methylated, low methylated, intermediately methylated or unmethylated nucleic acid molecules). The epigenetic state can also refer to the number of nucleotides with epigenetic modification. For example, if the epigenetic modification is DNA methylation, then an epigenetic state can refer to the number of methylated nucleotides of the nucleic acid molecules.

Enriched sample: As used herein, “enriched sample” refers to a sample that has been enriched for specific regions of interest. The sample can be enriched by selectively amplifying regions of interest or by using double stranded DNA/RNA probes (e.g. probes from Twist Biosciences) or single stranded DNA/RNA probes that can hybridize to nucleic acid molecules of interest (e.g., SureSelect® probes, Agilent Technol). In some embodiments, an enriched sample refers to a subset of the processed sample that is enriched, where the subset of the processed sample being enriched contains nucleic acid molecules from the sample of cell-free polynucleotides and from the first and/or second subset of the fragment size control molecules. In other embodiments, enriched sample refers to a subset of the third spike-in sample that is enriched, where the subset of the third spike-in sample being enriched contains nucleic acid molecules from the cell-free polynucleotides sample and from the first, second, and/or third subset of the fragment size control molecules

First spike-in sample: As used herein, “first spike-in sample” is a sample in which a first subset of the fragment size control molecules has been added to the sample of cell-free polynucleotides from a subject.

Fourth spike-in sample: As used herein, “fourth spike-in sample” refers to a sample in which a fourth subset of the fragment size control molecules has been added to a subset of the enriched sample.

Fragment size bias: As used herein, “fragment size bias” refers to any artificial bias on the fragment length or size of the nucleic acid molecules being analyzed in an assay. The artefact bias could be due to operator handling or due the reagents and/or steps used in the assay. This bias does not include the true biological bias on the fragment sizes observed between healthy and diseased subjects. The assay can involve one or more steps such as, but not limited to, liquid handling, extraction, library preparation, enrichment, wash/clean up and sequencing. In some embodiments, there can be a fragment size bias that is different from sample to sample. Based on the reaction conditions and procedures of each of these steps, recovery of the nucleic acid molecules belonging to a particular fragment size can be biased. In the fragmentome analysis, having an accurate quantitative measure of the cfDNA molecules of a particular size is useful in estimating the fragment length distribution and fragmentation pattern of the cell-free DNA. By estimating the fragment size bias in an assay, one can correct for the fragment size bias (artificial bias) introduced by each step in the assay workflow and have a better estimate of the original fragment size distribution (that reflects the true biological bias) of the cfDNA molecules.

Fragment size control molecules: As used herein, “fragment size control molecules” refer to a set of nucleic acid molecules that are added to a sample of polynucleotides to evaluate and/or optimize the analysis of nucleic acid molecules in a sample. The fragment size control molecules can have two regions—a fragment size region and an identifier region. In some embodiments, the fragment size control molecules have consist only a fragment size region. In some embodiments, the fragment size control molecules can have both fragment size region and identifier region. The set of fragment size control molecules can be classified into subset(s) of fragment size control molecules, and each subset of the fragment size control molecules can be added at one or more different steps in an assay in order to evaluate fragment length distribution, QC metrics and/or contamination of samples in any of the steps—extraction, library preparation, enrichment, and/or sequencing of polynucleotides sample. In some embodiments, the fragment size control molecules can be used to estimate how well the original ends of the polynucleotides in the sample are preserved through the assay workflow. The length of these fragment size control molecules is predetermined and can mimic the natural size distribution of cell-free DNA. Each subset of fragment size control molecules can further be classified into group(s) of fragment size control molecules based on the length of the fragment size region, and each group can have a fragment size region of different lengths. For example, a subset of fragment size control molecules can be classified into three groups based on the length of the fragment size region—a first group can have a fragment size region of 120 bp length, a second group can have a fragment size region of 160 bp length, and a third group can have a fragment size region of 320 bp length. In some embodiments, the fragment size control molecules can be synthetic molecules—i.e., can be synthesized in vitro with or without the use of enzymes. In some embodiments, the fragment size control molecules can have a non-naturally occurring nucleic acid sequence. In some embodiments, the fragment size control molecules can have a naturally occurring nucleic acid sequence. In some embodiments, the amplicons generated via amplifying a specific region (i.e., of a particular length) in any genome, plasmid or vector or a portion thereof can be used as fragment size control molecules. In some embodiments, the plasmids, vectors or any genome can be digested using restriction enzymes and the products of the digestion can be used as fragment size control molecules. In some embodiments, fragment size control molecules can have a nucleic acid sequence corresponding to a non-human genome. For example, these molecules can either have (i) a sequence corresponding to regions of lambda phage DNA, (ii) a non-naturally occurring sequence, and/or (iii) a combination of (i) and (ii). In some embodiments, the fragment size control molecules may comprise non-naturally occurring nucleotide analogs.

Fragment size region: As used herein, the term “fragment size region” refers to a region of the fragment size control molecule that represents the length of the fragment size control molecules. Each group of the fragment size control molecules differ in the length of the fragment size region. For example, a subset of fragment size control molecules can be classified into three groups based on the length of the fragment size region—a first group can have a fragment size region of 120 bp length, a second group can have a fragment size region of 160 bp length, and a third group can have a fragment size region of 320 bp length. The length of the fragment size region can be between 10 bp and 1000 bp.

Fragment size score: As used herein, “fragment size score” refers to a score that represents the recovery of the fragment size control molecules that belong to a particular group and a particular subset. The identity of the fragment size control molecules and the subset to which the fragment size control molecules belong, are maintained through the use of barcodes. In some embodiments, the fragment size score can be estimated based on the number of fragment size control molecules that belong to a particular group and a particular subset. In some embodiments, the fragment size score can be estimated based on the number of sequencing reads of the fragment size control molecules that belong to a particular group and a particular subset. In some embodiments, the fragment size score can be estimated for a particular group in a particular subset. In some embodiments, the fragment size score can be estimated for each of the group in every subset. In some embodiments, the fragment size score can be an overall score of the fragment size control molecules used in a particular subset (i.e., for all a single score that represents the fragment size control molecules within a subset) or an overall score of the fragment size control molecules added in one or more steps of the assay (i.e., a single score that represents the fragment size control molecules in one or more subsets added in the assay). In some embodiments, the fragment size score can be measured as a difference in the amount of a subset of fragment size control molecules added at a particular step to the amount of another subset of fragment size control molecules added at a different step and in some embodiments, the amount can be either in terms of mass (e.g., fg, pg, ng, μg), number of sequencing reads or number of fragment size molecules (belonging to a subset or belonging to a particular group). In some embodiments, the fragment size score can be measured as the recovery (fraction or percentage) of fragment size control molecules (belonging to a subset or belonging to a particular group), where the recovery can be calculated utilizing a orthogonal measurement of the relative abundance of the fragment size control molecules within a particular subset added to the sample. In some embodiments, the orthogonal measurement can be obtained from gel electrophoresis, qPCR, ddPCR or PCR-free sequencing. In some embodiments, the fragment size score can be measured as a difference in the amount of fragment size control molecules of a particular length to the amount of fragment size control molecules of a different length.

Fragment size threshold: As used herein, “fragment size threshold” refers to a predetermined threshold value or range used to evaluate the fragment size bias in the analysis of nucleic acid molecules in a sample. These thresholds can also be used to correct for any fragment length distribution in any of the steps such as extraction, library preparation, enrichment, wash/clean up and sequencing. In some embodiments, the fragment size threshold can be estimated for a particular group in a particular subset. In some embodiments, each group in a subset has a particular fragment size threshold. In some embodiments, the fragment size threshold can be estimated for each of the group in every subset. In some embodiments, the fragment size threshold can be an overall threshold of the fragment size control molecules used in a particular subset (i.e., for all a single threshold for the fragment size control molecules within a subset) or an overall threshold of the fragment size control molecules added in one or more steps of the assay (i.e., a single threshold for the fragment size control molecules in one or more subsets added in the assay). For example, a set of fragment size control molecules comprises two subsets—subset 1 and subset 2. If each subset comprises two groups—G11 and G12 (for subset 1) and G21 and G22 (for subset 2), then each of the four groups can have a fragment size score (S)—S11 for G11, S12 for G12, S21 for G21, and S22 for G22, based on the recovery of the fragment size control molecules. Each group in a subset can have a separate predetermined fragment size threshold. In this example, T11, T12, T21, and T22 are the fragment size thresholds for G11, G12, G21, and G22 respectively. The threshold can be in terms of percentage or fraction, and the threshold can be a threshold range instead of a particular threshold value. For the method to be considered a success, at least one of these fragment size scores should be within its corresponding fragment size threshold. In some embodiments, if each of the plurality of fragment size scores is within the corresponding fragment size threshold, then the method is classified as performing successfully.

Genomic region: As used herein, “genomic region” refers to any region (e.g., range of base pair locations) of a genome, e.g., an entire genome, a chromosome, a gene, or an exon. A genomic region may be a contiguous or a non-contiguous region. A “genetic locus” (or “locus”) can be a portion or entirety of a genomic region (e.g., a gene, a portion of a gene, or a single nucleotide of a gene).

Identifier region: As used herein, “identifier region” refers to a region of the fragment size control molecule that is used in distinguishing a fragment size control molecule from the other fragment size control molecules. The identifier region is also used to distinguish fragment size control molecules from one subset to those from another subset. The identifier region can have molecular barcodes. The identifier region can be present in one or both sides of the fragment size region. The identifier region can be either contiguous or non-contiguous. The molecular barcode serves as the identifier of a fragment size control molecule. The identifier region can have an additional region facilitating binding of one or more primers (primer binding sites). In some embodiments, the identifier region can also have additional flow cell binding sites, such as P5 and P7, which allow the fragment size control molecules to attach to the flow cell surface of the next-generation sequencer (e.g., Illumina sequencer). In some embodiments, the identifier region comprises barcodes that act as (i) a molecule identifier barcode, e.g., barcode that is used to identify each fragment size control molecule and differentiate one fragment size control molecule from another and (ii) a subset identifier barcode, e.g., barcode that is be used to identify the subset to which the fragment size control molecule belongs (i.e., whether the fragment size control molecule belongs to subset 1 or subset 2). The subset identifier barcode may be the same for all the fragment size control molecules in a subset, and the subset identifier barcode of one subset may be different from the subset identifier barcode of the other subsets. In some embodiments, the subset identifier barcode of one subset in one sample can be different from the subset identifier barcode of the corresponding subset in the other samples. For example, if the fragment size control molecules are used to evaluate the contamination of samples, the subset identifier barcode of one subset is different from the subset identifier barcode of that corresponding subset used in all the other samples within the same flow cell/batch. In some embodiments, the identifier region can comprise sample indices to distinguish fragment size control molecules of one sample from those of the other samples and sequences for attaching to the flow cells of the sequencer. For example, the identifier region of the subset of fragment size control molecules added after the enrichment step can comprises sample index sequences. In some embodiments, the identifier region can be attached to the fragment size region via ligation. In some embodiments, the identifier region can be added to the fragment size region via PCR.

Mutation: As used herein, “mutation” refers to a variation from a known reference sequence and includes mutations such as, for example, single nucleotide variants (SNVs), and insertions or deletions (indels). A mutation can be a germline or somatic mutation. In some embodiments, a reference sequence for purposes of comparison is a wildtype genomic sequence of the species of the subject providing a test sample, typically the human genome.

Neoplasm: As used herein, the terms “neoplasm” and “tumor” are used interchangeably. They refer to abnormal growth of cells in a subject. A neoplasm or tumor can be benign, potentially malignant, or malignant. A malignant tumor is a referred to as a cancer or a cancerous tumor.

Next-Generation Sequencing: As used herein, “next-generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next-generation sequencing techniques include, but are not limited to, sequencing by synthesis, sequencing by ligation, and sequencing by hybridization. In some embodiments, next-generation sequencing includes the use of instruments capable of sequencing single molecules.

Nucleic Acid Tag: As used herein, “nucleic acid tag” refers to a short nucleic acid (e.g., less than about 500 nucleotides, about 100 nucleotides, about 50 nucleotides, or about 10 nucleotides in length), used to distinguish nucleic acids from different samples (e.g., representing a sample index), or different nucleic acid molecules in the same sample (e.g., representing a molecular barcode), of different types, or which have undergone different processing. The nucleic acid tag comprises a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples comprising nucleic acids bearing different molecular barcodes and/or sample indexes in which the nucleic acids are subsequently being deconvolved by detecting (e.g., reading) the nucleic acid tags. Nucleic acid tags (molecular barcodes) can also be referred to as identifiers (e.g. molecule identifier, sample identifier). Additionally, or alternatively, nucleic acid tags can be used as molecule identifiers (e.g., to distinguish between different molecules or amplicons of different parent molecules in the same sample or sub-sample). This includes, for example, uniquely tagging different nucleic acid molecules in a given sample, or non-uniquely tagging such molecules. In the case of non-unique tagging applications, a limited number of tags (i.e., molecular barcodes) may be used to tag each nucleic acid molecule such that different molecules can be distinguished based on their endogenous sequence information (for example, start and/or stop positions where they map to a selected reference genome, a sub-sequence of one or both ends of a sequence, and/or length of a sequence) in combination with at least one molecular barcode. Typically, a sufficient number of different molecular barcodes are used such that there is a low probability (e.g., less than about a 10%, less than about a 5%, less than about a 1%, or less than about a 0.1% chance) that any two molecules may have the same endogenous sequence information (e.g., start and/or stop positions, subsequences of one or both ends of a sequence, and/or lengths) and also have the same molecular barcode.

Partitioning: As used herein, the “partitioning” and “epigenetic partitioning” are used interchangeably. It refers to separating or fractionating the nucleic acid molecules based on a characteristic (e.g. the level/degree of epigenetic modification) of the nucleic acid molecules. The partitioning can be physical partitioning of molecules. Partitioning can involve separating the nucleic acid molecules into groups or sets based on the level of epigenetic modification (i.e. epigenetic state). For example, the nucleic acid molecules can be partitioned based on the level of methylation of the nucleic acid molecules. In some embodiments, the methods and systems used for partitioning may be found in PCT Patent Application No. PCT/US2017/068329 which is incorporated by reference in its entirety.

Partitioned set: As used herein, “partitioned set” refers to a set of nucleic acid molecules partitioned into a set/group based on the differential binding affinity of the nucleic acid molecules to a binding agent. The binding agent binds preferentially to the nucleic acid molecules comprising nucleotides with epigenetic modification. For example, if the epigenetic modification is methylation, the binding agent can be a methyl binding domain (MBD) protein. In some embodiments, a partitioned set can comprise nucleic acid molecules belonging to a particular level/degree of epigenetic modification (i.e., epigenetic state). For example, the nucleic acid molecules can be partitioned into three sets: one set for highly methylated nucleic acid molecules (or hypermethylated nucleic acid molecules), which can be referred as hypermethylated partitioned set or hyper partitioned set, another set for low methylated nucleic acid molecules (or hypomethylated nucleic acid molecules), which can be referred as hypomethylated partitioned set or hypo partitioned set and a third set for intermediately methylated nucleic acid molecules, which can be referred as intermediately methylated partitioned set or intermediate partitioned set. In another example, the nucleic acid molecules can be partitioned based on the number of nucleotides with epigenetic modification—one partitioned set can have nucleic acid molecules with nine methylated nucleotides and another partitioned set can have unmethylated nucleic acid molecules (zero methylated nucleotides).

Polynucleotide: As used herein, “polynucleotide”, “nucleic acid”, “nucleic acid molecule”, or “oligonucleotide” refers to a linear polymer of nucleosides (including deoxyribonucleosides, ribonucleosides, or analogs thereof) joined by inter-nucleosidic linkages. Typically, a polynucleotide comprises at least three nucleosides. Oligonucleotides often range in size from a few monomeric units, e.g., 3-4, to hundreds of monomeric units. Whenever a polynucleotide is represented by a sequence of letters, such as “ATGCCTG”, it will be understood that the nucleotides are in 5′ 3′ order from left to right and that in the case of DNA, “A” denotes deoxyadenosine, “C” denotes deoxycytidine, “G” denotes deoxyguanosine, and “T” denotes deoxythymidine, unless otherwise noted. The letters A, C, G, and T may be used to refer to the bases themselves, to nucleosides, or to nucleotides comprising the bases.

Processing: As used herein, “processing” refers to a set of steps used to generate a library of nucleic acids that is suitable for sequencing. The set of steps can include, but are not limited to, partitioning, end repairing, addition of sequencing adapters, tagging, and/or PCR amplification of nucleic acids.

Processed sample: As used herein, “processed sample” refers to a sample that has been processed, as described elsewhere herein. In some embodiments, a processed sample can refer to a subset of the nucleic acids extracted from the first spike-in sample that is processed, where the subset of the nucleic acids extracted from the first spike-in sample contains nucleic acid molecules from the sample of cell-free polynucleotides and from the first subset of the fragment size control molecules. In other embodiments, a processed sample refers to a subset of the second spike-in sample that is processed, where the subset of the second spike-in sample being processed contains nucleic acid molecules from the sample of cell-free polynucleotides and from the first subset and second subset of the fragment size control molecules.

Quantitative measure: As used herein, “quantitative measure” refers to an absolute or relative measure. A quantitative measure can be, without limitation, a number, a statistical measurement (e.g., frequency, mean, median, standard deviation, or quantile), or a degree or a relative quantity (e.g., high, medium, and low). A quantitative measure can be a ratio of two quantitative measures. A quantitative measure can be a linear combination of quantitative measures. A quantitative measure may be a normalized measure.

Reference Sequence: As used herein, “reference sequence” refers to a known sequence used for purposes of comparison with experimentally determined sequences. For example, a known sequence can be an entire genome, a chromosome, or any segment thereof. In some embodiments, the reference sequence can be at least about 20, at least about 50, at least about 100, at least about 200, at least about 250, at least about 300, at least about 350, at least about 400, at least about 450, at least about 500, at least about 1000, or more than 1000 nucleotides. A reference sequence can align with a single contiguous sequence of a genome or chromosome or can include non-contiguous segments that align with different regions of a genome or chromosome. In some embodiments, reference sequence can be an entire genome. Examples of reference sequences include, for example, human genomes, such as, hG19 and hG38.

Sample: As used herein, “sample” means anything capable of being analyzed by the methods and/or systems disclosed herein.

Second spike-in sample: As used herein, “second spike-in sample” is a sample in which a second subset of the fragment size control molecules has been added to the nucleic acids extracted from the first spike-in sample, where the nucleic acids extracted from the first spike-in sample contain nucleic acid molecules from the sample of cell-free polynucleotides and from the first subset of the fragment size control molecules.

Sequencing: As used herein, “sequencing” refers to any of a number of technologies used to determine the sequence (e.g., the identity and order of monomer units) of a biomolecule, e.g., a nucleic acid such as DNA or RNA. Examples of sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof.In some embodiments, sequencing can be performer by a gene analyzer such as, for example, gene analyzers commercially available from Illumina, Inc., Pacific Biosciences, Inc., or Applied Biosystems/Thermo Fisher Scientific, among many others.

Sequence Information: As used herein, “sequence information” in the context of a nucleic acid polymer means the order and identity of monomer units (e.g., nucleotides, etc.) in that polymer.

Somatic Mutation: As used herein, the terms “somatic mutation” or “somatic variation” are used interchangeably. They refer to a mutation in the genome that occurs after conception. Somatic mutations can occur in any cell of the body except germ cells and accordingly, are not passed on to progeny.

Subject: As used herein, “subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual in need of therapy or suspected of needing therapy. The terms “individual” or “patient” are intended to be interchangeable with “subject.”

For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

Third spike-in sample: As used herein, “third spike-in sample” is a sample in which a third subset of the fragment size control molecules has been added to the processed sample.

### DETAILED DESCRIPTION

I. Overview

The fragment size distribution and fragmentation pattern in circulating cell-free DNA can provide information on the source of the cell-free DNA and also on disease levels. However, biases can be introduced to the fragment size distribution and fragmentation pattern through various processes of extraction and methods used to read out or analyze (e.g., library preparation for next-generation sequencing) the fragment lengths and patterns. Hence, it is essential to have controls that can be used to understand the sources of bias—at what steps in the process the bias occurs and also, to use these controls to correct for the bias or normalize for sample-to-sample and batch level fragment size biases.

The present disclosure provides methods, compositions, and systems for normalizing fragment length recovery; optimizing an assay to recover all/majority of the fragments of desired size and as a QC metric. Such methods may comprise using fragment size control molecules as a control that can be added to a sample of cell-free polynucleotides at different steps, such as from extraction through sequencing. These controls may have predetermined fragment lengths to mimic the natural size distribution of cell-free DNA. The identity of the fragment size control molecules added at different steps can be maintained by using different barcodes, so that one can keep track of the fragment size bias at a particular step. These control molecules can be used in many applications, such as, but not limited to the following: (i) monitor the fragment size bias, (ii) optimize the process to reduce the fragment size bias, (iii) correct or normalize the fragment size biases introduced during the process by analyzing the recovery of the fragment size control molecules, (iv) as a QC metric to assess the performance of the assay based on the recovery of the fragment size control molecules, (v) as a QC metric for analyzing any contamination of samples and (v) optimizing the assay to minimize any contamination.

Cancer formation and progression may arise from both genetic and epigenetic modifications of deoxyribonucleic acid (DNA). The present disclosure provides methods of analysis of epigenetic modifications of DNA, such as cell-free DNA (cfDNA). Such epigenetic analysis includes methylation and DNA fragment patterns discernable from measuring changes in fragment length distribution or the frequency fragment endpoints mapping to genomic locations. Such “fragmentome” analysis can be used alone or in combination with existing technologies to determine the presence or absence of a disease or condition, prognosis of a diagnosed disease or condition, therapeutic treatment of a diagnosed disease or condition, or predicted treatment outcome for a disease or condition. An example of such a disease or condition is cancer.

Circulating cell-free DNA (cfDNA) may be predominantly short DNA fragments (e.g., having lengths from about 100 to 400 base pairs, with a mode of about 165 bp) shed from dying tissue cells into bodily fluids such as peripheral blood (plasma or serum). Analysis of cfDNA may reveal, in addition to cancer-associated genetic variants, epigenetic footprints and signatures of phagocytic removal of dying cells, which may result in an aggregate nucleosomal occupancy profile of present malignancies (e.g., tumors) as well as their microenvironment components.

Components or factors that may contribute to a plasma fragmentome signal (e.g., a signal obtained from analysis of cfDNA fragments) include (i) cell death type and associated chromatin condensation events during dismantling of DNA, (ii) clearance mechanisms, which may involve various types of engulfment machinery regulated by an immune system of a subject, (iii) non-malignant variation in blood composition, which may be affected by an underlying combination of cell types in circulation, (iv) multiple sources or causes of non-malignant cell death in organs or tissues of a given type, and (v) heterogeneity of cell types within cancer, since malignant solid tumors include tumor-associated normal, epithelial, and stromal cells, immune cells, and vascular cells, any of all of which may contribute to and be represented in a cfDNA sample (e.g., which may be obtained from a bodily fluid of a subject).

Cell-free DNA in the form of histone-protected complexes can be released by various host cells including neutrophils, macrophages, eosinophils, as well as tumor cells. Circulating DNA typically has a short half-life (e.g., about 10 to 15 minutes), and the liver is typically the major organ where circulating DNA fragments are removed from blood circulation. The accumulation of cfDNA in the circulation may result from increased cell death and/or activation, impaired clearance of cfDNA, and/or decreases in levels of endogenous DNase enzymes. Cell-free DNA circulating in a subject's bloodstream may typically be packed into membrane-coated structures (e.g., apoptotic bodies) or complexes with biopolymers (e.g., histones or DNA-binding plasma proteins). The process of DNA fragmentation and subsequent trafficking may be analyzed for their effects on the characteristics of cell-free DNA signals as detected by fragmentome analysis.

In a cell nucleus (e.g., of a human), DNA typically exists in nucleosomes, which are organized into structures comprising about 145 base pairs (bp) of DNA wrapped around a core histone octamer. Electrostatic and hydrogen-bonding interactions of DNA and nucleosomes may result in energetically unfavorable bending of DNA over the protein surface. Such bending may be sterically prohibitive to other DNA-binding proteins and hence may serve to regulate access to DNA in a cell nucleus. Nucleosome positioning in a cell may fluctuate dynamically (e.g., over time and across various cell states and conditions), e.g., DNA unwrapping and rewrapping. Since a fragmentome signal may reflect nucleosome-protected DNA fragments that originated from a configuration influenced by nucleosomal units, nucleosome stability and dynamics may influence such a fragmentome signal. These nucleosome dynamics may stem from a variety of factors, such as: (i) ATP-dependent remodeling complexes, which may use the energy of ATP hydrolysis to slide the nucleosomes and exchange or evict histones from the chromatin fiber, (ii) histone variants, which may possess properties distinct from those of canonical histones and create localized specific domains within the chromatin fiber, (iii) histone chaperones, which may control the supply of free histones and cooperate with chromatin remodelers in histone deposition and eviction, (iv) post-translational modifications (PTMs) of histones (e.g., acetylation, methylation, phosphorylation, and ubiquitination), which may directly or indirectly influence chromatin structure and (v) transcription factors and active transcription by RNA polymerases.

Hence, fragmentation signals or patterns in cfDNA may be indicative of an aggregate cfDNA signal, stemming from multiple events related to heterogeneity in chromatin organization across the genome. Such chromatin organization may differ depending on factors such as global cellular identity, metabolic state, regional regulatory state, local gene activity in dying cells, and mechanisms of DNA clearance. Moreover, cell-free DNA fragmentome signals may be only partially attributed to underlying chromatin architecture of contributing cells. Such cfDNA fragmentome signals may be indicative of a more complex footprint of chromatin compaction during cell death and DNA protection from enzymatic digestion. Hence, chromatin maps specific to a given cell type or cell lineage type may only partially contribute to the inherent heterogeneity of DNA accessibility due to changes in nucleosome stability, conformation, and composition at various stages of cell death or debris trafficking. As a result, some nucleosomes may become preferentially present or not present in cell-free DNA (e.g., there may be a filtering mechanism which influences cfDNA clearance and releases into the blood circulation), which may depend on factors such as the mode and mechanism of death and cell corpse clearance.

A fragmentome signal may be generated in a cell and released as cfDNA into blood circulation as a result of nuclear DNA fragmentation during cell processes such as apoptosis and necrosis. Such fragmentation may be produced as a result of different nuclease enzymes acting on DNA in different stages of cells, resulting in sequence-specific DNA cleavage patterns which may be analyzed in cfDNA fragmentome signals. Classifying such cleavage patterns may be a clinically relevant marker of cell environments (e.g., tumor microenvironments, inflammation, disease states, tumorigenesis, etc.).

The present disclosure provides methods, compositions, and systems for evaluating and correcting the fragment size bias in the analysis of nucleic acid molecules in a sample of polynucleotides (in some embodiments, the polynucleotides can be cell-free polynucleotides). These methods can be used in various applications, such as prognosis, diagnosis, and/or for monitoring of a disease.

The analysis of nucleic acid molecules in a sample of polynucleotides can be optimized and corrected for fragment size bias from measuring the recovery of the fragment size control molecules. Fragment size control molecules can be synthetic nucleic acid molecules that have a predetermined fragment length. A set of fragment size control molecules can comprise nucleic acid molecules comprising at least one subset of fragment size control molecules, which is added to a sample of polynucleotides at a particular step. In some embodiments, the set of fragment size control molecules can comprise two or more subsets of fragment size control molecules, and each subset is added at a different step of the assay. Each subset comprises one or more groups of fragment size control molecules and each group has a different fragment length or size. For example, if there are three steps in an assay for which the fragment size bias has to be analyzed, the set of fragment size control molecules can comprise three subsets (S1, S2 and S3) of fragment size control molecules and each subset is added to the sample, prior to each step (i.e., S1 added prior to step 1, S2 added prior to step 2 and S3 added prior to step 3).

In some embodiments, the fragment size control molecules can be synthetic nucleic acid molecules. In some embodiments, the amplicons generated via amplifying a specific region (i.e., of a particular length) in any genome, plasmid or vector or a portion thereof can be used as fragment size control molecules. The sequence and the length of the fragment size control molecules can be already known prior to analysis. In some embodiments, fragment size control molecules are designed such that these molecules do not form any secondary structures. In some embodiments, the sequences of the fragment size control molecules are designed such that they do not overlap with any human genomic region. Hence, by adding the fragment size control molecules to the sample of polynucleotides and by tracking the fragment size control molecules in the subset, one can analyze the recovery of the fragment size control molecules and thereby estimate, in some embodiments, the fragment size bias.

Accordingly, in one aspect, the present disclosure provides a method analyzing nucleic acid molecules in a sample of polynucleotides, comprising: (a) adding a subset of fragment size control molecules to the nucleic acid molecules in the sample of polynucleotides, thereby producing a first spike-in sample; (b) extracting nucleic acids from the first spike-in sample; (c) processing at least a subset of the extracted nucleic acids, thereby producing a processed sample, wherein the processing comprises partitioning, tagging, and/or amplifying at least a subset of the first spike-in sample; (d) enriching for at least a subset of the processed sample, thereby producing an enriched sample; (e) sequencing at least a subset of the enriched sample to generate a plurality of sequence reads; and (f) analyzing the plurality of sequence reads to generate a plurality of fragment size scores of the subset of fragment size control molecules. In some embodiments, the sample of polynucleotides can be sample of cell-free polynucleotides (e.g., cell-free DNA).

In some embodiments, the method further comprises comparing the plurality of fragment size scores with a plurality of fragment size thresholds. In some embodiments, the method can be used for optimizing the analysis of nucleic acid molecules in the sample of polynucleotides based on the plurality of fragment size scores. In some embodiments, the method can be used for correcting for fragment size bias in the analysis of nucleic acid molecules in the sample of polynucleotides using the plurality of fragment size scores. In some embodiments, the method can be used a quality control (QC) metric, wherein the method is classified as (i) being a success, if at least one of the plurality of fragment size scores is within a corresponding fragment size threshold of the plurality of fragment size thresholds; or (ii) being unsuccessful, if at least one of the plurality of fragment size scores is not within the corresponding fragment size threshold of the plurality of fragment size thresholds.

FIG. lA is a schematic representation of a method for analyzing nucleic acid molecules in a sample of cell-free polynucleotides according to an embodiment of the disclosure. In some embodiments, a set of fragment size control molecules may comprise at least one subset of pre-determined fragment size control molecules. In some embodiments, the set of fragment size control molecules may comprise one subset of fragment size control molecules. In 101A, a subset (subset 1) of the fragment size control molecules is added to a sample of polynucleotides, whose fragment size bias can be analyzed, to generate a first spike-in sample prior to the extraction of polynucleotides. In some embodiments, fragment size control molecule is tagged with one or more tags or molecular barcodes that can help in identifying each individual fragment size control molecule (molecule identifier) within the subset and also the subset to which it belongs, i.e., subset 1 (subset identifier).

In some embodiments, a subset of fragment size control molecules may comprise one or more groups of fragment size control molecules. In some embodiments, the one or more groups of fragment size control molecules can comprise nucleic acid molecules of different length and/or different sequences. In some embodiments, a group of fragment size control molecules may comprise fragment size control molecules of the same length and the same sequence. In some embodiments, each group may comprise fragment size control molecules of the same length but different sequences.

In 102A, the nucleic acid molecules of the first spike-in sample are extracted. In 103A, the extracted nucleic acid molecules are processed to generate a library of nucleic acid molecules suitable for sequencing, including steps such as, but not limited to, end repairing, addition of sequencing adapters, tagging, and/or PCR amplification. In some embodiments, the processing of the extracted nucleic acid molecules to generate a library of nucleic acid molecules includes steps such as, but not limited to, partitioning, end repairing, addition of sequencing adapters, tagging, wash/clean up and/or PCR amplification. In 104A, the nucleic acids in the processed sample are enriched for (i) nucleic acid molecules in the sample of cell-free polynucleotides belonging to specific regions of interest and (ii) fragment size control molecules of subset 1. In 105A, the enriched sample is sequenced, so that the recovery of the fragment size control molecules in subset 1 can be analyzed. In 106A, the sequence reads generated from the sequencer are analyzed to measure the recovery of subset 1 fragment size control molecules. In some embodiments, the recovery of fragment size control molecules can be calculated utilizing a orthogonal measurement of the relative abundance of the fragment size control molecules within a particular subset added to the sample. In some embodiments, the orthogonal measurement can be obtained from gel electrophoresis, qPCR, ddPCR or PCR-free sequencing.

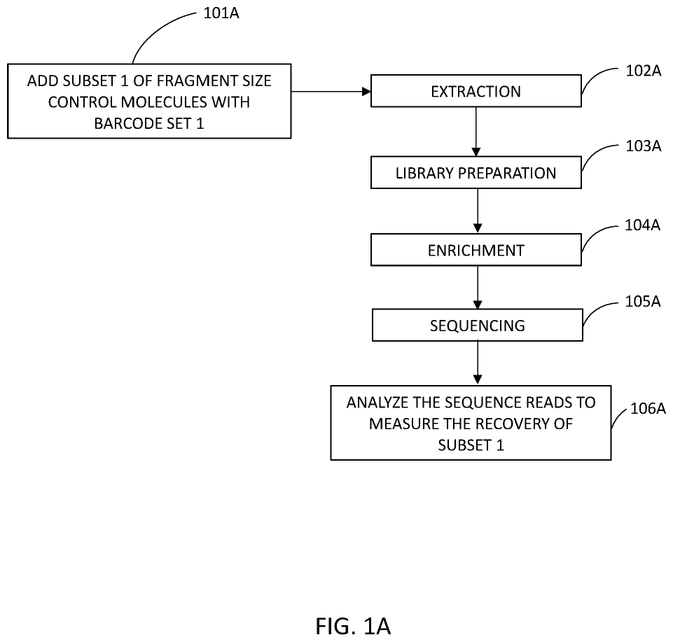

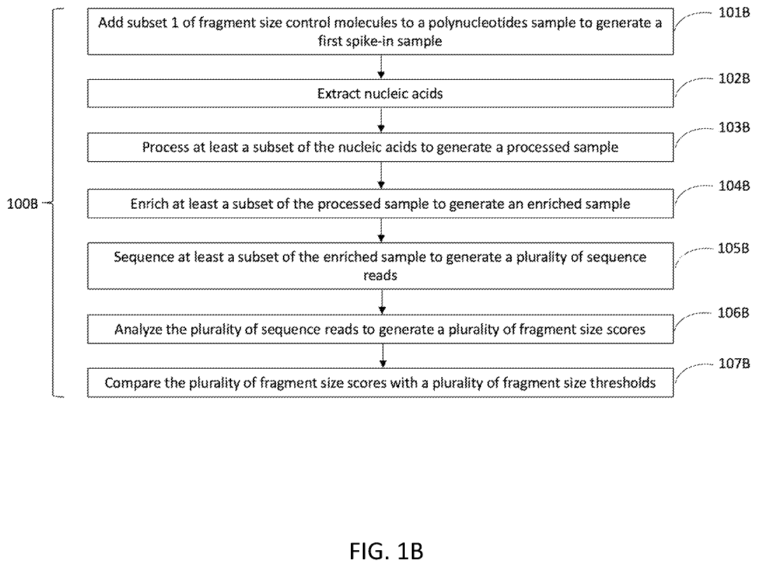

FIG. 1B illustrates an example embodiment of a method 100B for analyzing of nucleic acid molecules in a sample of polynucleotides (for example, the sample can be cell-free DNA sample). In 101B, a subset (subset 1) of the fragment size control molecules is added to a sample of polynucleotides, prior to the extraction step, to generate a first spike-in sample. Fragment size control molecules are added to the sample in order to monitor the fragment size bias which may be introduced in any of the steps. In some embodiments, the fragment size bias estimated using fragment size control molecules can be used to optimize the calculated fragment length distribution of polynucleotides in the sample. In some embodiments, a set of fragment size control molecules may comprise at least one subset of pre-determined fragment size control molecules. In some embodiments, a set of fragment size control molecules may comprise one subset (e.g., subset 1) of fragment size control molecules. In some embodiments, each subset comprises at least one group of fragment size control molecules. In some embodiments, each group comprises fragment size control molecules of the same length and the same sequence. In some embodiments, the length of the fragment size control molecules in one group is different from the length of the fragment size control molecules in the other groups. In some embodiments, each group comprises fragment size control molecules of the same length but different sequences. In some embodiments, fragment size control molecule is tagged with one or more tags or molecular barcodes that can help in identifying each individual fragment size control molecule (molecule identifier) within the subset and also the subset to which it belongs, i.e., subset 1 (subset identifier). In some embodiments, each ofthe subsets of fragment size control molecules is in equimolar concentration. In some embodiments, each of the subsets of fragment size control molecules is in non-equimolar concentration.

In 102B, the nucleic acid molecules of the first spike-in sample are extracted. In 103B, extracted nucleic acids are processed to generate a processed sample, wherein the processed sample comprises (i) nucleic acid molecules in the sample of cell-free polynucleotides and (ii) fragment size control molecules of subset 1.

The nucleic acid molecules are processed in 103B to generate a library of nucleic acids that is suitable for sequencing (e.g., by a next-generation sequencer). Processing can include steps such as, but not limited to, end repairing, addition of sequencing adapters, tagging, washing/clean up and/or amplification of nucleic acids. In some embodiments, processing can include steps such as, but not limited to, partitioning, end repairing, addition of sequencing adapters, tagging, washing/clean up and/or amplification of nucleic acids.

In some embodiments, the partitioning comprises partitioning the nucleic acid molecules based on a differential binding affinity of the nucleic acid molecules to a binding agent that preferentially binds to nucleic acid molecules comprising nucleotides with chemical modification (e.g., methylation). Examples of binding agents include, but are not limited to, methyl binding domains (MBDs) and methyl binding proteins (MBPs). Examples of MBPs contemplated herein include, but are not limited to:
(a) MeCP2, which is a protein preferentially binding to 5-methyl-cytosine over unmodified cytosine;
(b) RPL26, PRP8, and the DNA mismatch repair protein MHS6, which preferentially bind to 5-hydroxymethyl-cytosine over unmodified cytosine;
(c) FOXK1, FOXK2, FOXP1, FOXP4, AND FOXI3, which preferably bind to 5-formyl-cytosine over unmodified cytosine (e.g., as described by lurlaro et al., Genome Biol. 14, R119 (2013), which is hereby incorporated by reference in its entirety); and (d) Antibodies specific to one or more methylated nucleotide bases.

Partitioning can refer to separating or fractionating the nucleic acid molecules based on a characteristic of the nucleic acid molecules. The partitioning can be physical partitioning of molecules. Partitioning can involve separating the nucleic acid molecules into groups or sets based on the level of epigenetic modification (e.g., epigenetic state). For example, the nucleic acid molecules can be partitioned based on the level of methylation of the nucleic acid molecules. In some embodiments, the methods and systems used for partitioning may be found in PCT Patent Application No. PCT/US2017/068329, which is hereby incorporated by reference in its entirety. In those embodiments, the nucleic acids are partitioned based on the different levels of methylation (different number of methylated nucleotides). In some embodiments, the nucleic acids can be partitioned into two or more partitioned sets (e.g., at least 3, 4, 5, 6, or 7 partitioned sets). In some embodiments, the partitioned sets are representatives of nucleic acids having different extents of modifications (over representative or under representative of modifications). Over representation and under representation can be defined by the number of modifications born by a nucleic acid relative to the median number of modifications per strand in a population. For example, if the median number of 5-methylcytosine nucleotides in nucleic acid molecules in a sample is 2, a nucleic acid molecule including more than two 5-methylcytosine residues is over represented in this modification and a nucleic acid with 1 or zero 5-methylcytosine residues is under represented. The effect of the affinity separation is to enrich for nucleic acids over represented in a modification in a bound phase and for nucleic acids underrepresented in a modification in an unbound phase (i.e., in solution). The nucleic acids in the bound phase can be eluted before subsequent processing. In some embodiments, each of the plurality of partitioned sets is differentially tagged. The tagged partitioned sets are then pooled together for collective sample preparation, enrichment and/or sequencing. Differential tagging of the partitioned sets helps in keeping track of the nucleic acid molecules belonging to a particular partitioned set. The tags may be provided as components of adapters. The nucleic acid molecules in different partitioned sets receive different tags that can distinguish members of one partitioned set from another. The tags linked to nucleic acid molecules of the same partition set can be the same or different from one another. But if different from one another, the tags can have part of their sequence in common so as to identify the molecules to which they are attached as being of a particular partitioned set. For example, if the molecules of the spiked-in sample are partitioned into two partitioned sets—P1 and P2, then the molecules in P1 can be tagged with A1, A2, A3, and so forth, and the molecules in P2 can be tagged with B1, B2, B3, and so forth. Such a tagging system allows distinguishing the partitioned sets and between the molecules within a partitioned set.

In 104B, the processed sample is enriched to generate an enriched sample. The enriched sample comprises (i) nucleic acid molecules in the sample of cell-free polynucleotides belonging to specific regions of interest and (ii) fragment size control molecules of subset 1. In 105B, the enriched sample is sequenced to generate a plurality of sequence reads, so that the recovery of the fragment size control molecules in subset 1 can be analyzed, in order to calculate the fragment size bias. The sequence information obtained comprises sequence of the nucleic acid molecules and the tags attached to the nucleic acid molecules. From the sequence of the tags attached to the fragment size control molecules, one can correlate the tag with the individual fragment size control molecule and the subset to which the fragment size control molecule belongs. This information is used to analyze the recovery of the fragment size control molecules in the subset.

In 106B, the sequence reads are analyzed to generate fragment size scores of the fragment size control molecules. Fragment size score represents the recovery of the fragment size control molecules that belong to a particular group and a particular subset. The identity of the fragment size control molecules and the subset to which the fragment size control molecules belong, are maintained through the use of tags or barcodes. In some embodiments, the fragment size score can be estimated based on the number of fragment size control molecules that belong to a particular group and a particular subset. In some embodiments, the fragment size score can be estimated based on the number of sequencing reads of the fragment size control molecules that belong to a particular group and a particular subset. In some embodiments, the fragment size score can be estimated for a particular group in a particular subset. In some embodiments, the fragment size score can be estimated for each of the group in every subset. In some embodiments, the fragment size score can be an overall score of the fragment size control molecules used in a particular subset (i.e., for all a single score that represents the fragment size control molecules within a subset) or an overall score of the fragment size control molecules added in one or more steps of the assay (i.e., a single score that represents the fragment size control molecules in one or more subsets added in the assay). In some embodiments, the fragment size score can be measured as a difference in the amount of a subset of fragment size control molecules added at a particular step to the amount of another subset of fragment size control molecules added at a different step and in some embodiments, the amount can be either in terms of mass (e.g., fg, pg, ng, μg), number of sequencing reads or number of fragment size molecules (belonging to a subset or belonging to a particular group). In some embodiments, the fragment size score can be measured as the recovery (fraction or percentage) of fragment size control molecules (belonging to a subset or belonging to a particular group), where the recovery can be calculated utilizing a orthogonal measurement of the relative abundance of the fragment size control molecules within a particular subset added to the sample. In some embodiments, the orthogonal measurement can be obtained from gel electrophoresis, qPCR, ddPCR or PCR-free sequencing. In some embodiments, the fragment size score can be measured as a difference in the amount of fragment size control molecules of a particular length to the amount of fragment size control molecules of a different length.

In 107B, the fragment size scores are compared with the corresponding fragment size thresholds in order to determine the fragment size bias in the assay. Fragment size threshold is a predetermined threshold value or range used to evaluate or optimize the fragment size bias in the analysis of nucleic acid molecules in a sample. These thresholds can also be used to correct for any fragment length distribution in any of the steps such as extraction, library preparation, enrichment, washing/clean up and sequencing. In some embodiments, the fragment size threshold can be estimated for a particular group in a particular subset. In some embodiments, each group in a subset has a particular fragment size threshold. In some embodiments, the fragment size threshold can be estimated for each of the group in every subset. In some embodiments, the fragment size threshold can be an overall threshold of the fragment size control molecules used in a particular subset (i.e., for all a single threshold for the fragment size control molecules within a subset) or an overall threshold of the fragment size control molecules added in one or more steps of the assay (i.e., a single threshold for the fragment size control molecules in one or more subsets added in the assay). For example, a set of fragment size control molecules comprises two subsets—subset 1 and subset 2. If each subset comprises two groups—G11 and G12 (for subset 1) and G21 and G22 (for subset 2), then each of the four groups can have a fragment size score (S)—S11 for G11, S12 for G12, S21 for G21, and S22 for G22, based on the recovery of the fragment size control molecules. Each group in a subset can have a separate predetermined fragment size threshold. In this example, T11, T12, T21, and T22 are the fragment size thresholds for G11, G12, G21, and G22 respectively. The threshold can be in terms of percentage or fraction, and the threshold can be a threshold range instead of a particular threshold value. For the method to be considered a success, at least one of these fragment size scores should be within its corresponding fragment size threshold. In some embodiments, if each of the plurality of fragment size scores is within the corresponding fragment size threshold, then the method is classified as performing successfully. In some embodiments, the fragment size threshold can be in terms of percentage or fraction. In some embodiments, the fragment size threshold can be a threshold range instead of a particular threshold value.

In some embodiments, prior to 103B, a second subset (subset 2) of the fragment size control molecules may be added to the extracted nucleic acids to generate a second spike-in sample. In these embodiments, the second spike-in sample is processed to generate a processed sample. The processed sample comprises (i) nucleic acid molecules in the sample of cell-free polynucleotides and (ii) fragment size control molecules of subset 1 and subset 2. In some embodiments, prior to 104B, a third subset (subset 3) of the fragment size control molecules may be added to the processed sample to generate a third spike-in sample. In these embodiments, the third spike-in sample is enriched to generate an enriched sample. The enriched sample comprises (i) nucleic acid molecules in the sample of cell-free polynucleotides belonging to specific regions of interest and (ii) fragment size control molecules of subset 1, subset 2 and subset 3. In some embodiments, prior to 105B, a fourth subset (subset 4) of the fragment size control molecules may be added to the enriched sample to generate a fourth spike-in sample. The fourth spike-in sample comprises (i) nucleic acid molecules in the sample of cell-free polynucleotides belonging to specific regions of interest and (ii) fragment size control molecules of subset 1, subset 2, subset 3, and subset 4. In these embodiments, the fourth spike-in sample is sequenced to generate a plurality of sequence reads.

In another aspect, the present disclosure provides a method for analyzing nucleic acid molecules in a sample of polynucleotides, comprising: (a) adding a first subset of fragment size control molecules to the nucleic acid molecules in the sample of polynucleotides, thereby producing a first spike-in sample; (b) extracting nucleic acids from the first spike-in sample; (c) adding a second subset of fragment size control molecules to the extracted nucleic acids, thereby producing a second spike-in sample; (d) processing at least a subset of the second spike-in sample, thereby producing a processed sample, wherein the processing comprises partitioning, tagging, and/or amplifying the at least the subset of the second spike-in sample; (e) adding a third subset of fragment size control molecules to the processed sample, thereby producing a third spike-in sample; (f) enriching for at least a subset of the third spike-in sample, thereby producing an enriched sample; (g) adding a fourth subset of fragment size control molecules to at least a subset of the enriched sample, thereby producing a fourth spike-in sample; (h) sequencing the fourth spike-in sample to generate a plurality of sequence reads; (i) analyzing the plurality of sequence reads to generate a plurality of fragment size scores of the first subset of fragment size control molecules, the second subset of fragment size control molecules, the third subset of fragment size control molecules, and/or the fourth subset of fragment size control molecules; and (j) comparing the plurality of fragment size scores with a plurality of fragment size thresholds.

In some embodiments, the method further comprises comparing the plurality of fragment size scores with a plurality of fragment size thresholds. In some embodiments, the method can be used for optimizing the analysis of nucleic acid molecules in the sample of polynucleotides based on the plurality of fragment size scores. In some embodiments, the method can be used for correcting for fragment size bias in the analysis of nucleic acid molecules in the sample of polynucleotides using the plurality of fragment size scores. In some embodiments, the method can be used a quality control (QC) metric, wherein the method is classified as (i) being a success, if at least one of the plurality of fragment size scores is within a corresponding fragment size threshold of the plurality of fragment size thresholds; or (ii) being unsuccessful, if at least one of the plurality of fragment size scores is not within the corresponding fragment size threshold of the plurality of fragment size thresholds. In some embodiments, the sample of polynucleotides can be sample of cell-free polynucleotides (e.g., cell-free DNA).

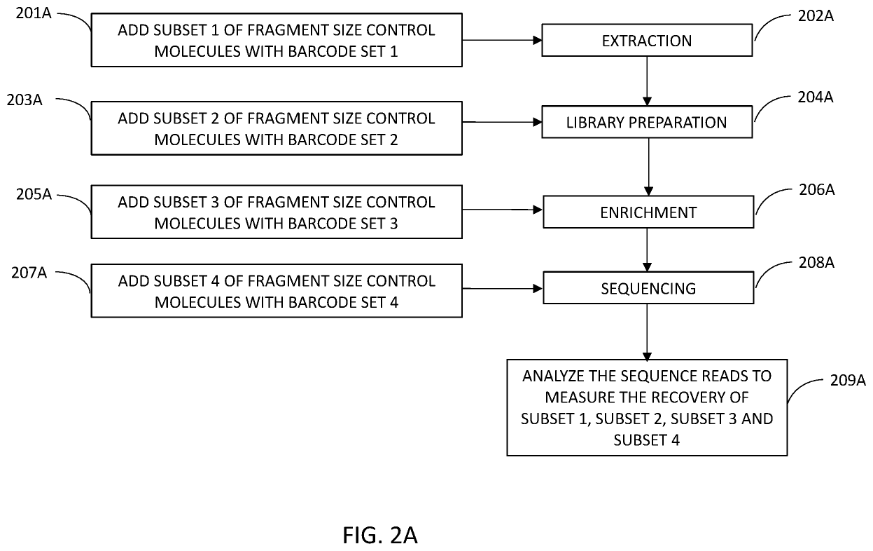

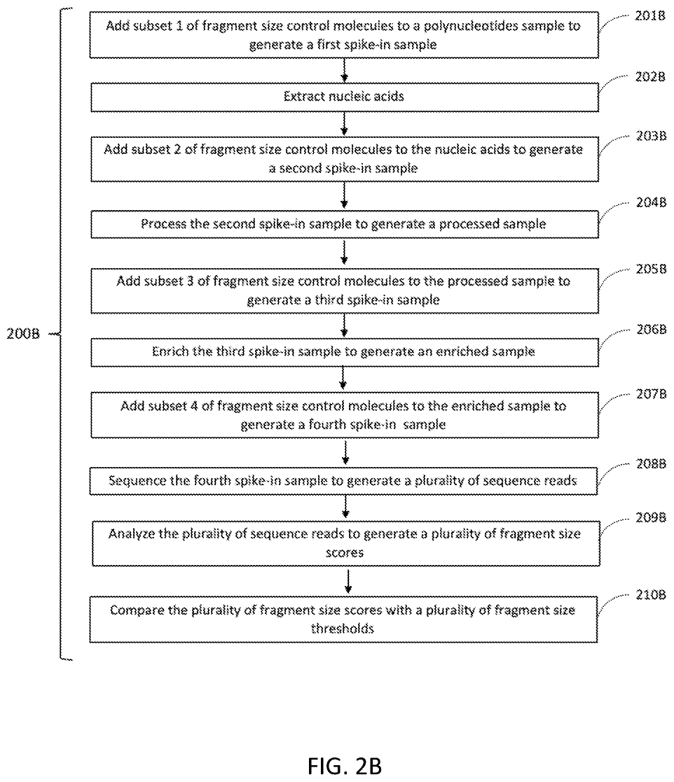

FIG. 2A is a schematic representation of a method for analyzing nucleic acid molecules in a sample of polynucleotides (e.g., cell-free polynucleotides) according to an embodiment of the disclosure. In some embodiments, a set of fragment size control molecules may comprise at least one subset of pre-determined fragment size control molecules. In 201A, a first subset (subset 1) of the fragment size control molecules is added to a sample of polynucleotides, whose fragment size bias has to be analyzed, to generate a first spike-in sample prior to the extraction of polynucleotides. In some embodiments, fragment size control molecule is tagged with one or more tags or barcodes that can help in identifying each individual fragment size control molecule (molecule identifier) within the subset and also the subset that it belongs to, i.e., subset 1 (subset identifier).

In some embodiments, a subset of fragment size control molecules may comprise one or more groups of fragment size control molecules. In some embodiments, the one or more groups of fragment size control molecules can comprise nucleic acid molecules of different length and/or different sequences. In some embodiments, a group of fragment size control molecules may comprise fragment size control molecules of same length and same sequence. In some embodiments, each group may comprise fragment size control molecules of same length but different sequences.

In 202A, the cell-free nucleic acid molecules of the first spike-in sample are extracted. In 203A, a second subset (subset 2) of the fragment size control molecules is added to the extracted nucleic acid molecules to generate a second spike-in sample. In 204A, the second spike-in sample is processed to generate a library of nucleic acid molecules suitable for sequencing, including steps such as, but not limited to, end repairing, addition of sequencing adapters, tagging, washing/clean up and/or PCR amplification. In some embodiments, the processing of the first spike-in sample to generate a library of nucleic acid molecules includes steps such as, but not limited to, partitioning, end repairing, addition of sequencing adapters, tagging, washing/clean up and/or PCR amplification. In 205A, a third subset (subset 3) of the fragment size control molecules is added to the processed sample to generate a third spike-in sample. In 206A, the nucleic acids in the third spike-in sample are enriched for (i) nucleic acid molecules in the sample of cell-free polynucleotides belonging to specific regions of interest and (ii) fragment size control molecules of subset 1, subset 2, and subset 3. In 207A, a fourth subset (subset 4) of the fragment size control molecules is added to the enriched sample to generate a fourth spike-in sample. In 208A, the fourth spike-in sample is sequenced, so that the recovery of the fragment size control molecules in subset 1, subset 2, subset 3, and subset 4 can be analyzed, in order to determine the fragment size bias. In 209A, the sequence reads generated from the sequencer are analyzed to measure the recovery of subset 1, subset 2, subset 3, and subset 4 fragment size control molecules.

FIG. 2B illustrates an example embodiment of a method 200B for analyzing nucleic acid molecules in a sample of polynucleotides (e.g., cell-free polynucleotides). In 201B, a subset (subset 1) of the fragment size control molecules is added to a sample of cell-free polynucleotides, prior to the extraction step, to generate a first spike-in sample. Fragment size control molecules are added to the sample in order to monitor and/or correct the fragment size bias introduced in any of the steps. In some embodiments, a set of fragment size control molecules may comprise at least one subset of pre-determined fragment size control molecules. In some embodiments, a set of fragment size control molecules may comprise one subset (e.g., subset 1) of fragment size control molecules. In some embodiments, each subset comprises at least one group of fragment size control molecules. In some embodiments, each group comprises fragment size control molecules of the same length and the same sequence. In some embodiments, the length of the fragment size control molecules in one group is different from the length of the fragment size control molecules in the other groups. In some embodiments, each group comprises fragment size control molecules of the same length but different sequences. In some embodiments, fragment size control molecule is tagged with one or more tags or barcodes that can help in identifying each individual fragment size control molecule (molecule identifier) within the subset and also the subset to which it belongs, i.e., subset 1 (subset identifier). In some embodiments, each of the subsets of fragment size control molecules is in equimolar concentration. In some embodiments, each of the subsets of fragment size control molecules is in non-equimolar concentration.

In 202B, the nucleic acid molecules of the first spike-in sample are extracted. In 203B, a second subset (subset 2) of the fragment size control molecules is added to the extracted nucleic acid molecules to generate a second spike-in sample. In some embodiments, fragment size control molecules in the first subset are differentiated from the fragment size control molecules in the second subset by using different tags i.e., the tags (subset identifier) of the fragment size control molecules in subset 1 are different from the tags of the fragment size control molecules in subset 2. For example, all fragment size control molecules in subset 1 may have the subset identifier tag (S1) and all fragment size control molecules in subset 2 may have the subset identifier tag (S2). Apart from the subset identifier tag, each fragment size control molecule comprises a molecule identifier tag that is used identifying each individual fragment size control molecule.

In 204B, the second spike-in sample is processed to generate a processed sample, wherein the processed sample comprises (i) nucleic acid molecules in the sample of cell-free polynucleotides and (ii) fragment size control molecules of subset 1 and subset 2. The second spike-in sample is processed in 204B to generate a library of nucleic acids that is suitable for sequencing (e.g., by a next-generation sequencer). Processing can include steps such as, but not limited to, end repairing, addition of sequencing adapters, tagging, washing/clean up and/or amplification of nucleic acids.

In some embodiments, processing can include steps such as, but not limited to, partitioning, end repairing, addition of sequencing adapters, tagging, washing/clean up and/or amplification of nucleic acids. In some embodiments, the partitioning comprises partitioning the nucleic acid molecules based on a differential binding affinity of the nucleic acid molecules to a binding agent that preferentially binds to nucleic acid molecules comprising nucleotides with chemical modification (e.g., methylation). In some embodiments, the nucleic acids can be partitioned into two or more partitioned sets (e.g., at least 3, 4, 5, 6, or 7 partitioned sets). In some embodiments, each of the plurality of partitioned sets is differentially tagged, such that the set of tags used in a first partitioned set of the plurality of partitioned sets is different from the set of tags used in a second partitioned set of the plurality of partitioned sets. The tagged partitioned sets are then pooled together for collective sample preparation, enrichment and/or sequencing. Differential tagging of the partitioned sets helps in keeping track of the nucleic acid molecules belonging to a particular partitioned set. The tags may, in some embodiments, be provided as components of adapters. The nucleic acid molecules in different partitioned sets receive different tags that can distinguish members of one partitioned set from another. The tags linked to nucleic acid molecules of the same partition set can be the same or different from one another. But if different from one another, the tags can have part of their sequence in common so as to identify the molecules to which they are attached as being of a particular partitioned set.

In some embodiments, the tagging comprises attaching a set of tags to the nucleic acids to produce a population of tagged nucleic acids, wherein the tagged nucleic acids comprise one or more tags. In some embodiments, the set of tags are attached to the nucleic acids by ligation of adapters to the nucleic acids, wherein the adapters comprise one or more tags.

In 205B, a third subset (subset 3) of the fragment size control molecules is added to the processed sample to generate a third spike-in sample. In 206B, the third spike-in sample is enriched to generate an enriched sample. The enriched sample comprises (i) nucleic acid molecules in the sample of cell-free polynucleotides belonging to specific regions of interest and (ii) fragment size control molecules of subset 1, subset 2, and subset 3. In 207B, a fourth subset (subset 4) of the fragment size control molecules is added to the enriched sample to generate a fourth spike-in sample. In 208B, the fourth spike-in sample is sequenced to generate a plurality of sequence reads, so that the recovery of the fragment size control molecules in subset 1, subset 2, subset 3, and subset 4 can be analyzed, in order to calculate the fragment size bias. The sequence information obtained comprises sequences of the nucleic acid molecules and the tags attached to the nucleic acid molecules. From the sequence of the tags attached to the fragment size control molecules, one can correlate the tag with the individual fragment size control molecule and the subset to which the molecule belongs. This information is used to analyze the recovery of the fragment size control molecules in the subset.

In 209B, the sequence reads are analyzed to generate fragment size scores of the fragment size control molecules. Fragment size score represents the recovery of the fragment size control molecules that belong to a particular group and a particular subset. In some embodiments, the recovery of fragment size control molecules can be calculated utilizing a orthogonal measurement of the relative abundance of the fragment size control molecules within a particular subset added to the sample. In some embodiments, the orthogonal measurement can be obtained from gel electrophoresis, qPCR, ddPCR or PCR-free sequencing. The identity of the fragment size control molecules and the group and the subset to which the fragment size control molecules belong, are maintained through the use of tags or barcodes. In some embodiments, the fragment size score can be estimated based on the number of fragment size control molecules that belong to a particular group and a particular subset. In some embodiments, the fragment size score can be estimated based on the number of sequencing reads of the fragment size control molecules that belong to a particular group and a particular subset. In some embodiments, the fragment size score can be estimated for a particular group in a particular subset. In some embodiments, the fragment size score can be estimated for each of the group in every subset. In some embodiments, the fragment size score can be an overall score of the fragment size control molecules used in a particular subset (i.e., a single score that represents the fragment size control molecules within a subset) or an overall score of the fragment size control molecules added in one or more steps of the assay (i.e., a single score that represents the fragment size control molecules in one or more subsets added in the assay). In some embodiments, the fragment size score can be measured as a difference in the amount of a subset of fragment size control molecules added at a particular step to the amount of another subset of fragment size control molecules added at a different step and in some embodiments, the amount can be either in terms of mass (e.g., fg, pg, ng, μg), number of sequencing reads or number of fragment size molecules (belonging to a subset or belonging to a particular group). In some embodiments, the fragment size score can be measured as the recovery (fraction or percentage) of fragment size control molecules (belonging to a subset or belonging to a particular group), where the recovery can be calculated utilizing a orthogonal measurement of the relative abundance of the fragment size control molecules within a particular subset added to the sample. In some embodiments, the orthogonal measurement can be obtained from gel electrophoresis, qPCR, ddPCR or PCR-free sequencing. In some embodiments, the fragment size score can be measured as a difference in the amount of fragment size control molecules of a particular length to the amount of fragment size control molecules of a different length.

In 210B, the fragment size scores are compared with the corresponding fragment size thresholds, in order to determine the fragment size bias in the assay. Fragment size threshold is a predetermined threshold value or range used to evaluate or optimize the fragment size bias in the analysis of nucleic acid molecules in a sample. These thresholds can also be used to correct for any fragment length distribution in any of the steps such as extraction, library preparation, enrichment, washing/clean up and sequencing. In some embodiments, the fragment size threshold can be estimated for a particular group in a particular subset. In some embodiments, each group in a subset has a particular fragment size threshold. In some embodiments, the fragment size threshold can be estimated for each of the group in every subset. In some embodiments, the fragment size threshold can be an overall threshold of the fragment size control molecules used in a particular subset (i.e., a single threshold for the fragment size control molecules within a subset) or an overall threshold of the fragment size control molecules added in one or more steps of the assay (i.e., a single threshold for the fragment size control molecules in one or more subsets added in the assay). For example, a set of fragment size control molecules comprises two subsets—subset 1 and subset 2. If each subset comprises two groups—G11 and G12 (for subset 1) and G21 and G22 (for subset 2), then each of the four groups can have a fragment size score (S)—S11 for G11, S12 for G12, S21 for G21, and S22 for G22, based on the recovery of the fragment size control molecules. Each group in a subset can have a separate predetermined fragment size threshold. In this example, T11, T12, T21, and T22 are the fragment size thresholds for G11, G12, G21, and G22 respectively. The threshold can be in terms of percentage or fraction, and the threshold can be a threshold range instead of a particular threshold value. For the method to be considered a success, at least one of these fragment size scores should be within its corresponding fragment size threshold. In some embodiments, if each of the plurality of fragment size scores is within the corresponding fragment size threshold, then the method is classified as performing successfully. In some embodiments, the fragment size threshold can be in terms of percentage or fraction. In some embodiments, the fragment size threshold can be a threshold range instead of a particular threshold value.

In some embodiments, the analysis of nucleic acid molecules for fragment size bias, which is estimated using fragment size control molecules can be used to optimize the calculated fragment length distribution of polynucleotides in the sample. In some embodiments, the plurality of fragment size scores is used to correct for fragment size bias in the analysis of nucleic acid molecules. In some embodiments, correcting the fragment size bias comprises using a quantitative measure that is derived from the ratio of fragment size score to fragment size threshold of a group within a subset and/or using a quantitative measure that is derived from the ratio of the fragment size scores of two groups within a subset and/or ratio of the fragment size scores of two subsets. In some embodiments, the method is classified as (i) being a success, if at least one of the plurality of fragment size scores is within a corresponding fragment size threshold of the plurality of fragment size thresholds; or (ii) being unsuccessful, if at least one of the plurality of fragment size scores is not within the corresponding fragment size threshold of the plurality of fragment size thresholds.

In some embodiments, if the fragment size scores of fragment size control molecules for all the groups in all the subsets is within the corresponding fragment size thresholds for all the groups, then the method of analyzing nucleic acid molecules may be classified as being a success. Otherwise, the method of analyzing nucleic acid molecules may be classified as being unsuccessful if any one of the fragment size scores is outside of its corresponding fragment size threshold. For example, a set of fragment size control molecules comprises two subsets of fragment size control molecules—subset 1 and subset 2. If each subset comprises two groups—G11 and G12 (for subset 1) and G21 and G22 (for subset 2), then each of the four groups can have a fragment size score (S)—S11 for G11, S12 for G12, S21 for G21, and S22 for G22, for based on the recovery of the fragment size control molecules. Each group in a subset has a separate fragment size threshold. In this example, T11, T12, T21, and T22 are the fragment size thresholds for G11, G12, G21, and G22 respectively. If S11<T11, S12<T12, S21<T21, and S22<T22, then the method may be classified as being successful. If any one of the fragment size scores is outside of (i.e., not within) its corresponding fragment size threshold, then the method may be classified as being unsuccessful.

In some embodiments, fragment size control molecules may be used within individual samples to correct for fragment size bias in the observed fragment length distributions resulting from technical (artificial bias) rather than true biological causes. For example, because cfDNA molecules derived from tumor cells typically exhibit a shorter average length than do fragments derived from hematopoietic cells, summary statistics, including but not limited to the mean, median, mode, and IQR, of the fragment length distribution may be used as a feature for detection of malignancy, either alone or in combination with other features. However, these summary statistics may be impacted by technical factors—artificial bias introduced during shipment, sample and liquid handling and any of the steps (e.g., extraction, partitioning, tagging, amplification, washing/clean up, enrichment and sequencing), and these impacts may confound the detection of malignancy. To avoid this potential source of confounding, the observed fragment length distribution may be adjusted based on the relative recovery of fragment size control molecules of different lengths. For example, if the expected recovery of fragment size control molecules of a particular length is 75%, but the observed recovery of fragment size control molecules of that particular length in a given sample is only 25%, the density of the component of the fragment length distribution of sample polynucleotides corresponding to the length of the fragment size control molecules may be increased three-fold to correct for the low recovery. Conversely, if the expected recovery of fragment size control molecules of a particular length is 60% but the observed recovery of fragment size control molecules of that particular length in a given sample is 80%, the fragment length distribution's density of the sample polynucleotides may be decreased by 25% corresponding to the length of the fragment size control molecules.

The adjustment to the observed fragment length distribution may occur in a window around the length of the recovered fragment size control molecules. For example, if the fragment size control molecules have length of 200 base-pairs, the observed length distribution density may be adjusted for sample polynucleotides with length between 180 bp and 220 bp, or 170 bp and 230 bp. The window need not be symmetrical, and the applied adjustment need not be the same for every length of the polynucleotides falling within the window. As an example, the applied adjustment may use a Gaussian kernel centered on the length of the fragment size control molecules rather than a uniform kernel.

The expected recovery of fragment size control molecules of different lengths can be determined by performing one or more control experiments. Fragment size thresholds may be set for the recovery of any fragment size control molecules of a particular length. If the observed recovery of the fragment size control molecules fall below a fragment size threshold value or not within a fragment size threshold range, the sample may be considered to have failed quality control. These fragment size thresholds may differ for fragment size control molecules of different lengths.

In another aspect, the present disclosure provides a method for producing a sequencing library of a sample of cell-free polynucleotides, comprising: (a) adding a subset of fragment size control molecules to the sample, thereby producing a first spike-in sample; (b) extracting nucleic acids from the first spike-in sample; (c) processing at least a subset of the extracted nucleic acids, thereby producing a processed sample, wherein the processing comprises partitioning, tagging and/or amplifying at least a subset of the first spike-in sample; and (d) enriching for at least a subset of the processed sample. In some embodiments, the method further comprises, prior to the processing, adding a second subset of fragment size control molecules, thereby producing a second spike-in sample. In some embodiments, the method further comprises, prior to the enriching, adding a third subset of fragment size control molecules, thereby producing a third spike-in sample. In some embodiments, the method further comprises, e) adding a fourth subset of fragment size control molecules, thereby producing a fourth spike-in sample.

In another aspect, the present disclosure provides a method for detecting contamination of a first sample with a second sample, comprising, for each first sample and second sample: (a) adding a subset of fragment size control molecules to generate a first spiked-in sample, wherein the subset of fragment size control molecules added to the first sample can be distinguished from the subset of fragment size control molecules added to the second sample; b) extracting nucleic acids from the first spike-in; (c) processing at least a subset of the extracted nucleic acids, thereby producing a processed sample, wherein the processing comprises partitioning, tagging and/or amplifying at least a subset of the first spike-in sample; (d) enriching for at least a subset of the processed sample; (e) sequencing at least a subset of the enriched sample to generate a plurality of sequence reads; and (f) analyzing the plurality of sequence reads to generate one or more contamination scores of the subset of fragment size control molecules. In some embodiments, the method further comprises, prior to the processing, adding a second subset of fragment size control molecules, thereby producing a second spike-in sample, wherein the subset of fragment size control molecules added to the first sample can be distinguished from the subset of fragment size control molecules added to the second sample. In some embodiments, the method further comprises, prior to the enriching, adding a third subset of fragment size control molecules, thereby producing a third spike-in sample, wherein the subset of fragment size control molecules added to the first sample can be distinguished from the subset of fragment size control molecules added to the second sample. In some embodiments, the method further comprises, prior to the sequencing, adding a fourth subset of fragment size control molecules, thereby producing a fourth spike-in sample., wherein the subset of fragment size control molecules added to the first sample can be distinguished from the subset of fragment size control molecules added to the second sample. In some embodiments, the subset of fragment size control molecules added to the first sample can be distinguished from the subset of fragment size control molecules added to the second sample by using a subset identifier barcodes in the first sample that is different from the subset identifier barcodes used in the other samples.

Contamination score refers to a score in a first sample that represents the presence of fragment size control molecules added to a second sample. In some embodiments, the subset identifier barcode used in one subset in one sample can be different from the subset identifier used in the other subsets in the other samples. In these embodiments, from the sequence of the subset identifier barcode present in the fragment size control molecules, the presence of fragment size control molecules that belong to a different second sample can be identified. In some embodiments, the contamination score can be specific for each type/group of fragment size control molecules used in a subset (i.e., a separate contamination score for different lengths/groups of fragment size control molecules used in a subset) or it could be an overall score that represents the different lengths/groups of fragments size control molecules. In some embodiments, the contamination score can be estimated based on the number of fragment size control molecules that belong to a different second sample. In some embodiments, the contamination score can be estimated based on the number of sequencing reads of the fragment size control molecules that belong to a different second sample. In some embodiments, the contamination score can be estimated based on the number of sequencing reads of the fragment size control molecules that belong to a different second sample. In some embodiments, the contamination score of a sample can be estimated based on the fraction or percentage of number of sequencing reads of the fragment size control molecules that belong to other samples to the number of sequencing reads of the fragment size control molecules added to that sample. In some embodiments, the contamination score of a sample can be estimated based on the fraction or percentage of number of molecules of the fragment size control molecules that belong to other samples to the number of molecules of the fragment size control molecules added to that sample. In these embodiments, the fragment size control molecules added to that molecule can be identified from the subset identifier barcode of the fragment size control molecules.

In some embodiments, the method further comprises comparing at least one or more contamination scores with at least one or more contamination thresholds. Contamination threshold refers to a predetermined threshold value or range used to evaluate the contamination of samples in the analysis of nucleic acid molecules in a sample. These thresholds can also be used to optimize the assay in any of the steps such as extraction, library preparation, enrichment, washing/clean up and sequencing. In some embodiments, the contamination threshold can be specific for each type/group of fragment size control molecules used in a subset (i.e., a separate contamination threshold for different lengths/groups of fragment size control molecules used in a subset). In some embodiments, the contamination threshold can be an overall threshold for the different lengths/groups of fragments size control molecules in a subset or an overall threshold for the fragment size control molecules used in one or more subsets added in the assay. In some embodiments, each group in a subset has a particular contamination threshold. For example, a set of fragment size control molecules comprises two subsets—subset 1 and subset 2. If each subset comprises two groups—G11 and G12 (for subset 1) and G21 and G22 (for subset 2), then each of the four groups can have a contamination score (S)—S11 for G11, S12 for G12, S21 for G21, and S22 for G22, based on the recovery of the fragment size control molecules. Each group in a subset can have a separate predetermined contamination threshold. In this example, T11, T12, T21, and T22 are the contamination thresholds for G11, G12, G21, and G22 respectively. The threshold can be in terms of percentage or fraction, and the threshold can be a threshold range instead of a particular threshold value. For the method to be considered a success, at least one of these contamination scores should be within its corresponding contamination threshold. In some embodiments, if each of the plurality of contamination scores is within the corresponding contamination threshold, then the method is classified as performing successfully. In some embodiments, the method further comprises classifying the first sample as (i) being contaminated with the second sample, if at least one or more of the contamination scores is not within a corresponding contamination threshold of the one or more contamination thresholds; or (ii) being not contaminated with the second sample, if at least one or more of the contamination scores is within the corresponding contamination threshold of the one or more contamination thresholds.

II. Fragment Size Control Molecules

Fragment size control molecules are nucleic acid molecules that are added to a sample of polynucleotides to evaluate and/or optimize the analysis of nucleic acid molecules in a sample. The fragment size control molecules can have two regions—a fragment size region and an identifier region. The set of fragment size control molecules can be classified into subset(s) of fragment size control molecules, and each subset of the fragment size control molecules can be added at one or more different steps in an assay in order to evaluate fragment length distribution, QC metrics and/or contamination of samples in any of the steps—extraction, library preparation, enrichment, and/or sequencing of cell-free polynucleotide sample. The length of these fragment size control molecules is predetermined and in some embodiments, can mimic the natural size distribution of cell-free DNA. Each subset of fragment size control molecules can further be classified into group(s) of fragment size control molecules based on the length of the fragment size region, and each group can have a fragment size region of different lengths. For example, a subset of fragment size control molecules can be classified into three groups based on the length of the fragment size region—a first group can have a fragment size region of 120 bp length, a second group can have a fragment size region of 160 bp length, and a third group can have a fragment size region of 320 bp length. In some embodiments, the fragment size control molecules can be synthetic oligonucleotides. In some embodiments, the fragment size control molecules can have a non-naturally occurring nucleic acid sequence. In some embodiments, the fragment size control molecules can have a naturally occurring nucleic acid sequence. In some embodiments, the amplicons generated via amplifying a specific region (i.e., of a particular length) in any genome, plasmid or vector or a portion thereof can be used as fragment size control molecules. In some embodiments, fragment size control molecules can have a nucleic acid sequence corresponding to a non-human genome. For example, these molecules can either have (i) a sequence corresponding to regions of lambda phage DNA, (ii) a non-naturally occurring sequence, and/or (iii) a combination of (i) and (ii). In some embodiments, the fragment size control molecules may comprise non-naturally occurring nucleotide analogs

In another aspect, the present disclosure provides a set of fragment size control molecules, comprising at least one subset of pre-determined fragment size control molecules, wherein the at least one subset of pre-determined fragment size control molecules comprises a plurality of fragment size control molecules comprising a fragment size region. In some embodiments, the fragment size control molecules further comprise an identifier region. Fragment size region is a region of the fragment size control molecule that represents the length of the fragment size control molecules. In some embodiments, the at least one subset comprises at least one group of fragment size control molecules. In some embodiments, the fragment size region of the fragment size control molecules in a group are of same length. Each group of the fragment size control molecules can differ in the length of the fragment size region. For example, a subset of fragment size control molecules can be classified into three groups based on the length of the fragment size region—a first group can have a fragment size region of 120 bp length, a second group can have a fragment size region of 160 bp length, and a third group can have a fragment size region of 320 bp length. The length of the fragment size region can be between 10 bp and 1000 bp. In some embodiments, the length of the fragment size region in a group is different from the length of the fragment size region in the other groups.

The identifier region is a region of the fragment size control molecule that is used in distinguishing a fragment size control molecule from the other fragment size control molecules. The identifier region is also used to distinguish fragment size control molecules from one subset to those from another subset. In some embodiments, the identifier region is on one or both sides of the fragment size region. In some embodiments, the identifier region comprises a molecular barcode. The molecular barcode serves as the identifier of a fragment size control molecule. The identifier region can be present in one or both sides of the fragment size region. The molecular barcode serves as the identifier of a fragment size control molecule. In some embodiments, the identifier region comprises barcodes that act as (i) a molecule identifier barcode, e.g., barcode that is used to identify each fragment size control molecule and differentiate one fragment size control molecule from another and (ii) a subset identifier barcode, e.g., barcode that is be used to identify the subset to which the fragment size control molecule belongs (i.e., whether the fragment size control molecule belongs to subset 1 or subset 2). The subset identifier barcode may be the same for all the fragment size control molecules in a subset, and the subset identifier barcode of one subset may be different from the subset identifier barcode of the other subsets. For example, all fragment size control molecules in subset 1 can have a subset identifier tag Si, and all fragment size control molecules in subset 2 can have a subset identifier tag S2. In some embodiments, the subset identifier barcode of one subset in one sample can be different from the subset identifier barcode of the corresponding subset in the other samples. For example, if the fragment size control molecules are used to evaluate the contamination of samples, the subset identifier barcode of one subset is different from the subset identifier barcode of that corresponding subset used in all the other samples within the same flow cell/batch. In some embodiments, the identifier region can comprise sample index to distinguish fragment size control molecules of one sample from those of the other samples. For example, the identifier region of the subset of fragment size control molecules added after the enrichment step can comprises sample index sequences. In some embodiments the identifier region can be attached to the fragment size region via ligation.

In some embodiments, the fragment size control molecules comprise one or more primer binding sites. In some embodiments, the primer binding site is in the identifier region. In some embodiments, the identifier region can also have additional flow cell binding sites, such as P5 and P7, which allow the fragment size control molecules to attach to the flow cell surface of the next-generation sequencer (e.g., Illumina sequencer).

In some embodiments, the fragment size region of the fragment size control molecules in a group comprises the same oligonucleotide sequence. In some embodiments, the fragment size region of the fragment size control molecules in a group comprises at least two distinguishable oligonucleotide sequences. In some embodiments, the fragment size region of the fragment size control molecules in the first subset comprises an oligonucleotide sequence distinguishable from the oligonucleotide sequence of the fragment size region of the fragment size control molecules in the second subset.

In some embodiments, the fragment size region can be at least 10 bp, at least 50 bp, at least 60 bp, at least 70 bp, at least 80 bp, at least 90 bp, at least 100 bp, at least 120 bp, at least 150 bp, at least 200 bp, at least 250 bp, at least 300 bp, at least 400 bp, at least 500 bp, at least 600 bp, at least 700 bp, at least 800 bp, at least 900 bp, or at least 1000 bp in length. In some embodiments, the length of the fragment size region can be between 10 bp and 1000 bp. In some embodiments, each of the subsets of fragment size control molecules is in equimolar concentration. In some embodiments, each of the subsets of fragment size control molecules is in non-equimolar concentration. In some embodiments, each of the groups of fragment size control molecules in the subset is in equimolar concentration. In some embodiments, each of the groups of fragment size control molecules in the subset is in non-equimolar concentration.

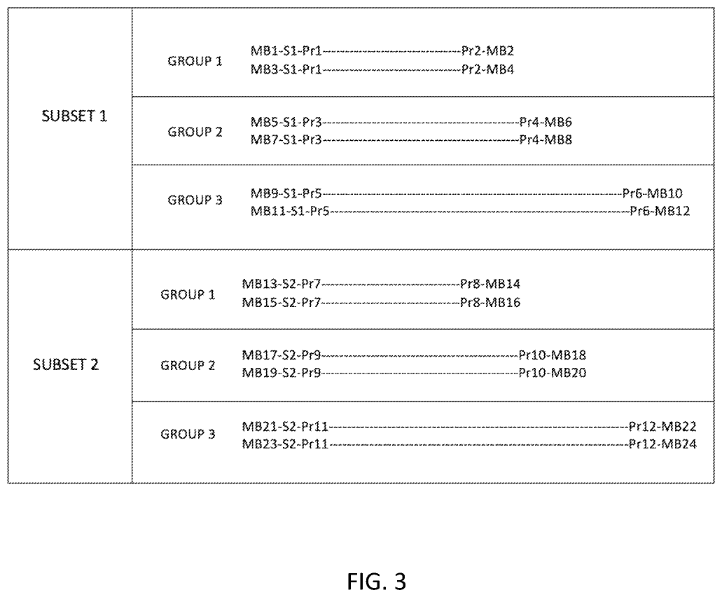

FIG. 3 is a schematic representation of fragment size control molecules suitable for use with some embodiments of the disclosure. The set of fragment size control molecules described here have a length similar to that of the natural size distribution of cell-free DNA. In FIG. 3, as an example, the set of fragment size control molecules have been categorized into two subsets—subset 1 and subset 2. The fragment size control molecules in FIG. 3 are double-stranded DNA molecules. For illustration purposes, only one strand of the double-stranded fragment size control molecule is shown. In this embodiment, each subset is categorized into three groups—group 1, group 2, and group 3. In FIG. 3, the fragment size region of all fragment size control molecules within each group have the same sequence, and the length of the fragment size region in one group is different from the length of the fragment size region in the other groups. In FIG. 3, the “------” region represents the fragment size region of the fragment size control molecule. In this embodiment, the identifier region is present on both sides of the fragment size region. The identifier region has primer binding sites on both ends of the fragment size region. Also, here, the sequence of the fragment size region of group 1 in subset 1 is the same as the sequence of the fragment size region of group 1 in subset 2. Likewise, the sequence of the fragment size region of group 2 and group 3 in subset 1 is the same as the sequence of the fragment size region of group 2 and group 3 in subset 2, respectively.

The identifier region on both sides of the fragment size region have molecule identifier barcode (MB) whereas the subset identifier barcode (S) is on one side only. Molecular barcode is used as an identifier of individual fragment size control molecules, and each fragment size control molecule has a unique molecular barcode (i.e., molecule 1 has MB1 & MB2, molecule 2 has MB3 & MB4, molecule 3 has MB5 & MB6, and so forth). A subset identifier barcode may be used as an identifier of the subset to which the fragment size control molecule belongs. Here, all the fragment size control molecules of subset' and subset 2 have a subset identifier barcode of S1 and S2, respectively. In this example, the subset identifier barcode is on one side of the fragment size region.

In some embodiments, the molecular barcode can be on one or both sides of the fragment size region. In some embodiments, the subset identifier barcode can be on one or both sides of the fragment size region.

In this embodiment, the identifier region has primer binding sites on both sides of the fragment size region. Here, for subset 1, fragment size control molecules of group 1, group 2, and group 3 have Pr1 & Pr2, Pr3 & Pr4, and Pr5 & Pr6 primer binding sites, respectively, on both sides of the fragment size region. In some embodiments, the identifier region can have an additional region facilitating binding of one or more primers (primer binding sites). In some embodiments, the primer binding sites of the identifier region in one subset is different from the primer binding sites in the other subsets. In some embodiments, these primer binding sites are used in analyzing the recovery of the fragment size control molecules. In some embodiment, instead of analyzing the recovery of the fragment size control molecules by sequencing, the recovery of the fragment size control molecules can be analyzed by digital droplet PCR (ddPCR), quantitative qPCR or gel electrophoresis using primers that bind to these primer binding sites.

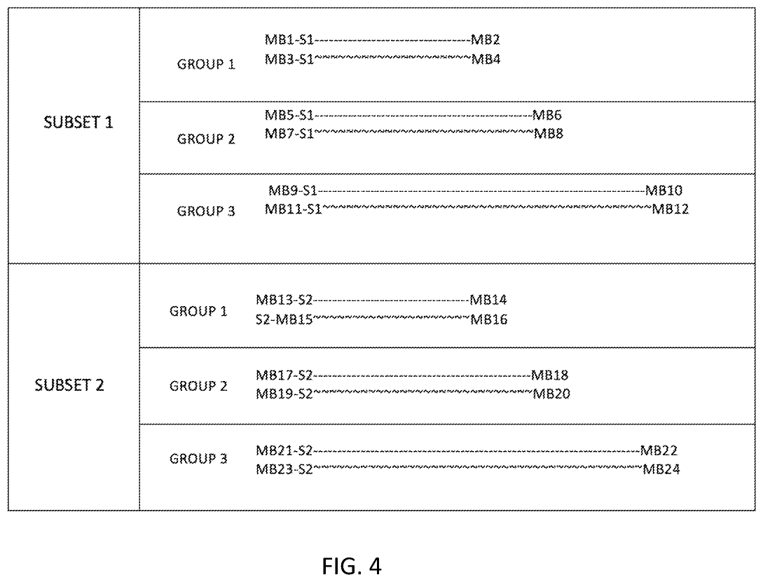

FIG. 4 is a schematic representation of fragment size control molecules suitable for use with some embodiments of the disclosure. In FIG. 4, as an example, the set of fragment size control molecules have been categorized into two subsets—subset 1 and subset 2. The fragment size control molecules in FIG. 4 are double-stranded DNA molecules. For illustration purposes, only one strand of the double-stranded fragment size control molecule is shown. In this embodiment, each subset is categorized into three groups—group 1, group 2 and group 3. In FIG. 4, the fragment size region of all fragment size control molecules within each group are of same length but the sequence of the fragment size region within a group can be different. In FIG. 4, the “------” and “ [symbol] ” regions represent the fragment size region of the fragment size control molecule. The “------” and “ [symbol] ” region show that each group comprises two different sequences, but they are of same length. The length of the fragment size region in one group is different from the length of the fragment size region in the other groups. In this embodiment, the identifier region is present on both sides of the fragment size region. Also, here, the two sequences of the fragment size region of group 1 in subset 1 are the same as the two sequences of the fragment size region of group 1 in subset 2. Likewise, the two sequences of the fragment size region of group 2 and group 3 in subset 1 are the same as the two sequences of the fragment size region of group 2 and group 3 in subset 2, respectively.

The identifier regions on both sides have molecule identifier barcode (MB) whereas the subset identifier barcode (S) is on one side only. The molecular barcode is used as an identifier of individual fragment size control molecules and each fragment size control molecule has a unique molecular barcode (i.e., molecule 1 has MB1 & MB2, molecule 2 has MB3 & MB4, molecule 3 has MB5 & MB6, and so forth). A subset identifier barcode may be used as an identifier of the subset to which the fragment size control molecule belongs. Here, all the fragment size control molecules of subset 1 and subset 2 have a subset identifier barcode of S1 and S2, respectively. In this example, the subset identifier barcode is on one side of the fragment size region.

In some embodiments, the molecular barcode can be on one or both sides of the fragment size region. In some embodiments, the subset identifier barcode can be on one or both sides of the fragment size region.

In some embodiments, the fragment size control molecules can have a non-naturally occurring nucleic acid sequence. In some embodiments, the fragment size control molecules can have a naturally occurring nucleic acid sequence. In some embodiments, fragment size control molecules can have a nucleic acid sequence corresponding to a non-human genome. For example, these molecules can either have (i) a sequence corresponding to regions of lambda phage DNA, (ii) a non-naturally occurring sequence, and/or (iii) a combination of (i) and (ii). In some embodiments, the fragment size control molecules may comprise non-naturally occurring nucleotide analogs.

In some embodiments, the sample of polynucleotides is a sample of DNA, a sample of RNA, a sample of cell-free polynucleotides, a sample of cell-free DNA, or a sample of cell-free RNA. In some embodiments, the sample of polynucleotides is a sample of cell-free DNA.

In some embodiments, the cell-free DNA is at least at least 1 ng, at least 5 ng, at least 10 ng, at least 15 ng, at least 20 ng, at least 30 ng, at least 50 ng, at least 75 ng, at least 100 ng, at least 150 ng, at least 200 ng, at least 250 ng, at least 300 ng, at least 350 ng, at least 400 ng, at least 450 ng, or at least 500 ng.

In some embodiments, the amount of fragment size control molecules is at least 1 attomole, at least 2 attomoles, at least 5 attomoles, at least 10 attomoles, at least 15 attomoles, at least 20 attomoles, at least 50 attomoles, at least 75 attomoles, at least 100 attomoles, at least 1 femtomole, at least 2 femtomoles, at least 5 femtomoles, at least 10 femtomoles, at least 15 femtomoles, at least 20 femtomoles, at least 50 femtomoles, at least 75 femtomoles, at least 100 femtomoles, at least 125 femtomoles, at least 150 femtomoles, or at least 200 femtomoles at least 300 femtomoles, at least 400 femtomoles, at least 500 femtomoles, at least 600 femtomoles, at least 700 femtomoles, at least 800 femtomoles, at least 900 femtomoles, at least 1 picomole, at least 2 picomoles, at least 5 picomoles, or at least 10 picomoles. In some embodiments, the amount of fragment size control molecules can be between 1 attomole and 10 picomoles.

Additional embodiments of the present disclosure include composition to which the size fragment control molecules have been added. For example, a cell-free DNA sample to which size fragment control molecules is an embodiment of the present disclosure. Similarly, the numerous compositions comprising one or more different subsets of size fragment control molecules produced during the practice of the subject methods are considered embodiments of the present disclosure.

III. General Features of the Methods
A. Samples

A sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, and urine. Samples may be bodily fluids, such as blood and fractions thereof, and urine. Such samples can include nucleic acids shed from tumors. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a bodily fluid for analysis can be plasma or serum containing cell-free nucleic acids, e.g., cell-free DNA (cfDNA).

In some embodiments, the sample volume of bodily fluid taken from a subject depends on the desired read depth for sequenced regions. Examples of volumes are about 0.4-40 milliliters (mL), about 5-20 mL, about 10-20 mL. For example, the volume can be about 0.5 mL, about 1 mL, about 5 mL, about 10 mL, about 20 mL, about 30 mL, about 40 mL, or more milliliters. A volume of sampled plasma is typically between about 5 mL to about 20 mL.

The sample can comprise various amounts of nucleic acid. Typically, the amount of nucleic acid in a given sample is equates with multiple genome equivalents. For example, a sample of about 30 nanograms (ng) DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

In some embodiments, a sample comprises nucleic acids from different sources, e.g., from cells and from cell-free sources (e.g., blood samples, etc.). Typically, a sample includes nucleic acids carrying mutations. For example, a sample optionally comprises DNA carrying germline mutations and/or somatic mutations. Typically, a sample comprises DNA carrying cancer-associated mutations (e.g., cancer-associated somatic mutations).

Example amounts of cell-free nucleic acids in a sample before amplification typically range from about 1 femtogram (fg) to about 1 microgram (μg), e.g., about 1 picogram (pg) to about 200 nanograms (ng), about 1 ng to about 100 ng, about 10 ng to about 1000 ng. In some embodiments, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In some embodiments, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some embodiments, methods include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides length (in samples from human subjects) and a second minor peak in a range between about 240 nucleotides to about 440 nucleotides in length. In some embodiments, cell-free nucleic acids are from about 160 nucleotides to about 180 nucleotides in length, or from about 320 nucleotides to about 360 nucleotides in length, or from about 440 nucleotides to about 480 nucleotides in length.

In some embodiments, cell-free nucleic acids are isolated from bodily fluids through a partitioning step in which cell-free nucleic acids, as found in solution, are separated from intact cells and other non-soluble components of the bodily fluid. In some embodiments, partitioning includes techniques such as centrifugation or filtration. Alternatively, cells in bodily fluids may be lysed, and cell-free and cellular nucleic acids may be processed together. Generally, after addition of buffers and wash steps, cell-free nucleic acids may be precipitated with, for example, an alcohol. In some embodiments, additional clean-up steps are used, such as silica-based columns to remove contaminants or salts. Non-specific bulk carrier nucleic acids, for example, are optionally added throughout the reaction to optimize aspects of the example procedure, such as yield. After such processing, samples typically include various forms of nucleic acids including double-stranded DNA, single-stranded DNA and/or single-stranded RNA. Optionally, single-stranded DNA and/or single-stranded RNA are converted to double-stranded forms so that they are included in subsequent processing and analysis steps.

B. Tagging

In some embodiments, the nucleic acid molecules (from the sample of polynucleotides and fragment size control molecules) may be tagged with sample indexes and/or molecular barcodes (referred to generally as “tags”). Tags may be incorporated into or otherwise joined to adapters by chemical synthesis, ligation (e.g., blunt-end ligation or sticky-end ligation), or overlap extension polymerase chain reaction (PCR), among other methods. Such adapters may be ultimately joined to the target nucleic acid molecule. In other embodiments, one or more rounds of amplification cycles (e.g., PCR amplification) are generally applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after sequence capturing steps are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based capturing steps. In some embodiments, the sample indexes are introduced after sequence capturing steps are performed. In some embodiments, molecular barcodes are incorporated to the nucleic acid molecules (e.g. cfDNA molecules) in a sample through adapters via ligation (e.g., blunt-end ligation or sticky-end ligation). In some embodiments, sample indexes are incorporated to the nucleic acid molecules (e.g. cfDNA molecules) in a sample through overlap extension polymerase chain reaction (PCR). Typically, sequence capturing protocols involve introducing a single-stranded nucleic acid molecule complementary to a targeted nucleic acid sequence, e.g., a coding sequence of a genomic region and mutation of such region is associated with a cancer type.

In some embodiments, the tags may be located at one end or at both ends of the sample nucleic acid molecule. In some embodiments, tags are predetermined or random or semi-random sequence oligonucleotides. In some embodiments, the tags may be less than about 500, 200, 100, 50, 20, 10, 9, 8, 7, 6, 5, 4, 3, 2, or 1 nucleotides in length. The tags may be linked to sample nucleic acids randomly or non-randomly.

In some embodiments, each sample is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual molecules such that the combination of the molecular barcode and the sequence it may be attached to creates a unique sequence that may be individually tracked. Detection of non-uniquely tagged molecular barcodes in combination with endogenous sequence information (e.g., the beginning (start) and/or end (stop) genomic location/position corresponding to the sequence of the original nucleic acid molecule in the sample, sub-sequences of sequence reads at one or both ends, length of sequence reads, and/or length of the original nucleic acid molecule in the sample) typically allows for the assignment of a unique identity to a particular molecule. In some embodiments, detection of non-uniquely tagged molecular barcodes in combination with endogenous sequence information (e.g., the beginning (start) and/or end (stop) region of the alignment of the sequence reads to the reference sequence, sub-sequences of sequence reads at one or both ends, length of sequence reads, and/or length of the original nucleic acid molecule in the sample) typically allows for the assignment of a unique identity to a particular molecule. In some embodiments, the beginning region comprises a genomic start position of the sequencing read at which the 5′ end of the sequencing read is determined to start aligning to reference sequence and the end region comprises a genomic stop position of the sequencing read at which the 3′ end of the sequencing read is determined to stop aligning to the reference sequence. In some embodiments, beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence.

The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the target molecule) can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes.

In some embodiments, the assignment of unique or non-unique molecular barcodes in reactions is performed using methods and systems described in, for example, U.S. Patent Application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths).

A subset identifier barcode can be part of the identifier region of the fragment size control molecules. The subset identifier barcode is used to identify the subset to which the fragment size control molecule belongs (i.e., whether the fragment size control molecule belongs to subset 1 or subset 2). The subset identifier barcode may be the same for all the fragment size control molecules in a subset, and the subset identifier barcode of one subset may be different from the subset identifier barcode of the other subsets in the same sample and/or in the other samples used in the same batch as well. In some embodiments, the subset identifier barcode of one subset in one sample can be different from the subset identifier barcode of the corresponding subset in the other samples. For example, if the fragment size control molecules are used to evaluate the contamination of samples, the subset identifier barcode of one subset is different from the subset identifier barcode of that corresponding subset used in all the other samples within the same flow cell/batch. In some embodiments, the identifier region can comprise sample index to distinguish fragment size control molecules of one sample from those of the other samples. For example, the identifier region of the subset of fragment size control molecules added after the enrichment step can comprises sample index sequences.

In some embodiments, the assignment of unique or non-unique molecular barcodes in reactions is performed using methods and systems described in, for example, U.S. Patent Application Nos. 20010053519, 20030152490, and 20110160078, and U.S. Pat. Nos. 6,582,908, 7,537,898, 9,598,731, and 9,902,992 each of which is hereby incorporated by reference in its entirety.

C. Amplification

Sample nucleic acids and fragment size control molecules may be flanked by adapters and amplified by PCR and other amplification methods using nucleic acid primers binding to primer binding sites in adapters flanking a DNA molecule to be amplified. In some embodiments, amplification methods involve cycles of extension, denaturation, and annealing resulting from thermocycling, or can be isothermal as, for example, in transcription mediated amplification. Other examples of amplification methods that may be optionally utilized include the ligase chain reaction, strand displacement amplification, nucleic acid sequence-based amplification, and self-sustained sequence-based replication.

Typically, the amplification reactions generate a plurality of non-uniquely or uniquely tagged nucleic acid amplicons with molecular barcodes and sample indexes at size ranging from about 150 nucleotides (nt), to about 700 nt, from 250 nt to about 350 nt, or from about 320 nt to about 550 nt.

In some embodiments, the amplicons have a size of about 180 nt. In some embodiments, the amplicons have a size of about 200 nt.

D. Enrichment

In some embodiments, sequences are enriched prior to sequencing the nucleic acids. Enrichment optionally performed for specific target regions or nonspecifically (“target sequences”). In some embodiments, targeted regions of interest may be enriched with nucleic acid capture probes (“baits”) selected for one or more bait set panels using a differential tiling and capture scheme. A differential tiling and capture scheme generally uses bait sets of different relative concentrations to differentially tile (e.g., at different “resolutions”) across genomic regions associated with the baits, subject to a set of constraints (e.g., sequencer constraints such as sequencing load, utility of each bait, etc.), and capture the targeted nucleic acids at a desired level for downstream sequencing. These targeted genomic regions of interest optionally include natural or synthetic nucleotide sequences of the nucleic acid construct. In some embodiments, biotin-labeled beads with probes to one or more regions of interest can be used to capture target sequences, and optionally followed by amplification of those regions, to enrich for the regions of interest.

Sequence capture typically involves the use of oligonucleotide probes that hybridize to the target nucleic acid sequence. In some embodiments, a probe set strategy involves tiling the probes across a region of interest. Such probes can be, for example, from about 60 to about 120 nucleotides in length. The set can have a depth (e.g., depth of coverage) of about 2×, 3×, 4×, 5×, 6×, 7×, 8×, 9×, 10×, 15×, 20×, 50×, or more than 50×. The effectiveness of sequence capture generally depends, in part, on the length of the sequence in the target molecule that is complementary (or nearly complementary) to the sequence of the probe.

E. Sequencing

Sample nucleic acids and fragment size control molecules, optionally flanked by adapters, with or without prior amplification are generally subjected to sequencing. Sequencing methods or commercially available formats that are optionally utilized include, for example, Sanger sequencing, high-throughput sequencing, pyrosequencing, sequencing-by-synthesis, single-molecule sequencing, nanopore-based sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLiD, Ion Torrent, or Nanopore platforms. Sequencing reactions can be performed in a variety of sample processing units, which may include multiple lanes, multiple channels, multiple wells, or other means of processing multiple sample sets substantially simultaneously. Sample processing units can also include multiple sample chambers to enable the processing of multiple runs simultaneously.

The sequencing reactions can be performed on one or more nucleic acid fragment types or regions containing markers of cancer or of other diseases. The sequencing reactions can also be performed on any nucleic acid fragment present in the sample. The sequence reactions may be performed on at least about 5%, 10%, 15%, 20%, 25%, 30%, 40%, 50%, 60%, 70%, 80%, 90%, 95%, 99%, 99.9%, or 100% of the genome. In other cases, sequence reactions may be performed on less than about 5%, 10%, 15%, 20%, 25%, 30%, 40%, 50%, 60%, 70%, 80%, 90%, 95%, 99%, 99.9%, or 100% of the genome.

Simultaneous sequencing reactions may be performed using multiplex sequencing techniques. In some embodiments, cell-free polynucleotides are sequenced with at least about 1000, 2000, 3000, 4000, 5000, 6000, 7000, 8000, 9000, 10000, 50000, or 100,000 sequencing reactions. In other embodiments, cell-free polynucleotides are sequenced with less than about 1000, 2000, 3000, 4000, 5000, 6000, 7000, 8000, 9000, 10000, 50000, or 100,000 sequencing reactions. Sequencing reactions are typically performed sequentially or simultaneously. Subsequent data analysis is generally performed on all or part of the sequencing reactions. In some embodiments, data analysis is performed on at least about 1000, 2000, 3000, 4000, 5000, 6000, 7000, 8000, 9000, 10000, 50000, or 100,000 sequencing reactions. In other embodiments, data analysis may be performed on less than about 1000, 2000, 3000, 4000, 5000, 6000, 7000, 8000, 9000, 10000, 50000, or 100,000 sequencing reactions. An example of a read depth is from about 1000 to about 50000 reads per locus (e.g., base position).

F. Analysis

Sequencing may generate a plurality of sequence reads or reads. Sequence reads or reads may include sequences of nucleotide data less than about 150 bases in length, or less than about 90 bases in length. In some embodiments, reads are between about 80 bases and about 90 bases, e.g., about 85 bases in length. In some embodiments, methods of the present disclosure are applied to very short reads, e.g., less than about 50 bases or about 30 bases in length. Sequence read data can include the sequence data as well as meta information. Sequence read data can be stored in any suitable file format including, for example, VCF files, FASTA files, or FASTQ files.

FASTA may refer to a computer program for searching sequence databases, and the name FASTA may also refer to a standard file format. FASTA is described by, for example, Pearson & Lipman, 1988, Improved tools for biological sequence comparison, PNAS 85:2444-2448, which is hereby incorporated by reference in its entirety. A sequence in FASTA format begins with a single-line description, followed by lines of sequence data. The description line is distinguished from the sequence data by a greater-than (“>”) symbol in the first column. The word following the “>” symbol is the identifier of the sequence, and the rest of the line is the description (both are optional). There may be no space between the “>” and the first letter of the identifier. It is recommended that all lines of text be shorter than 80 characters. The sequence ends if another line starting with a “>” appears; this indicates the start of another sequence.

The FASTQ format is a text-based format for storing both a biological sequence (usually nucleotide sequence) and its corresponding quality scores. It is similar to the FASTA format but with quality scores following the sequence data. Both the sequence letter and quality score are encoded with a single ASCII character for brevity. The FASTQ format is a de facto standard for storing the output of high throughput sequencing instruments such as the Illumina Genome Analyzer, as described by, for example, Cock et al. (“The Sanger FASTQ file format for sequences with quality scores, and the Solexa/Illumina FASTQ variants,” Nucleic Acids Res 38(6):1767-1771, 2009), which is hereby incorporated by reference in its entirety.

For FASTA and FASTQ files, meta information includes the description line and not the lines of sequence data. In some embodiments, for FASTQ files, the meta information includes the quality scores. For FASTA and FASTQ files, the sequence data begins after the description line and is present typically using some subset of IUPAC ambiguity codes optionally with “−”. In an embodiment, the sequence data may use the A, T, C, G, and N characters, optionally including “−” or U as-needed (e.g., to represent gaps or uracil).

In some embodiments, the at least one master sequence read file and the output file are stored as plain text files (e.g., using encoding such as ASCII; ISO/IEC 646; EBCDIC; UTF-8; or UTF-16). A computer system provided by the present disclosure may include a text editor program capable of opening the plain text files. A text editor program may refer to a computer program capable of presenting contents of a text file (such as a plain text file) on a computer screen, allowing a human to edit the text (e.g., using a monitor, keyboard, and mouse). Examples of text editors include, without limitation, Microsoft Word, emacs, pico, vi, BBEdit, and TextWrangler. The text editor program may be capable of displaying the plain text files on a computer screen, showing the meta information and the sequence reads in a human-readable format (e.g., not binary encoded but instead using alphanumeric characters as they may be used in print or human writing).

While methods have been discussed with reference to FASTA or FASTQ files, methods and systems of the present disclosure may be used to compress any suitable sequence file format including, for example, files in the Variant Call Format (VCF) format. A typical VCF file may include a header section and a data section. The header contains an arbitrary number of meta-information lines, each starting with characters ‘##’, and a TAB delimited field definition line starting with a single ‘#’ character. The field definition line names eight mandatory columns and the body section contains lines of data populating the columns defined by the field definition line. The VCF format is described by, for example, Danecek et al. (“The variant call format and VCF tools,” Bioinformatics 27(15):2156-2158, 2011), which is hereby incorporated by reference in its entirety. The header section may be treated as the meta information to write to the compressed files and the data section may be treated as the lines, each of which can be stored in a master file only if unique.

Some embodiments provide for the assembly of sequence reads. In assembly by alignment, for example, the sequence reads are aligned to each other or aligned to a reference sequence. By aligning each read, in turn to a reference genome, all of the reads are positioned in relationship to each other to create the assembly. In addition, aligning or mapping the sequence read to a reference sequence can also be used to identify variant sequences within the sequence read. Identifying variant sequences can be used in combination with the methods and systems described herein to further aid in the diagnosis or prognosis of a disease or condition, or for guiding treatment decisions.

In some embodiments, any or all of the steps are automated. Alternatively, methods of the present disclosure may be embodied wholly or partially in one or more dedicated programs, for example, each optionally written in a compiled language such as C++, then compiled and distributed as a binary. Methods of the present disclosure may be implemented wholly or in part as modules within, or by invoking functionality within, existing sequence analysis platforms. In some embodiments, methods of the present disclosure include a number of steps that are all invoked automatically responsive to a single starting cue (e.g., one or a combination of triggering events sourced from human activity, another computer program, or a machine). Thus, the present disclosure provides methods in which any or the steps or any combination of the steps can occur automatically responsive to a cue. “Automatically” generally means without intervening human input, influence, or interaction (e.g., responsive only to original or pre-cue human activity).

The methods of the present disclosure may also encompass various forms of output, which includes an accurate and sensitive interpretation of a subject's nucleic acid sample. The output of retrieval can be provided in the format of a computer file. In some embodiments, the output is a FASTA file, a FASTQ file, or a VCF file. The output may be processed to produce a text file, or an XML file containing sequence data such as a sequence of the nucleic acid aligned to a sequence of the reference genome. In other embodiments, processing yields output containing coordinates or a string describing one or more mutations in the subject nucleic acid relative to the reference genome. Alignment strings may include Simple UnGapped Alignment Report (SUGAR), Verbose Useful Labeled Gapped Alignment Report (VULGAR), and Compact Idiosyncratic Gapped Alignment Report (CIGAR) (as described by, for example, Ning et al., Genome Research 11(10): 1725-9, 2001, which is hereby incorporated by reference in its entirety). These strings may be implemented, for example, in the Exonerate sequence alignment software from the European Bioinformatics Institute (Hinxton, UK).

In some embodiments, a sequence alignment is produced—such as, for example, a sequence alignment map (SAM) or binary alignment map (BAM) file—comprising a CIGAR string (the SAM format is described, e.g., by Li et al., “The Sequence Alignment/Map format and SAMtools,” Bioinformatics, 25(16):2078-9, 2009, which is hereby incorporated by reference in its entirety). In some embodiments, CIGAR displays or includes gapped alignments one-per-line. CIGAR is a compressed pairwise alignment format reported as a CIGAR string. A CIGAR string may be useful for representing long (e.g., genomic) pairwise alignments. A CIGAR string may be used in SAM format to represent alignments of reads to a reference genome sequence.

A CIGAR string may follow an established motif. Each character is preceded by a number, giving the base counts of the event. Characters used can include M, I, D, N, and S (M=match; I=insertion; D=deletion; N=gap; S=substitution). The CIGAR string defines the sequence of matches and/or mismatches and deletions (or gaps). For example, the CIGAR string 2MD3M2D2M may indicate that the alignment contains 2 matches, 1 deletion (number 1 is omitted in order to save some space), 3 matches, 2 deletions, and 2 matches.

In some embodiments, a nucleic acid population is prepared for sequencing by enzymatically forming blunt-ends on double-stranded nucleic acids with single-stranded overhangs at one or both ends. In these embodiments, the population is typically treated with an enzyme having a 5′-3′ DNA polymerase activity and a 3′-5′ exonuclease activity in the presence of the nucleotides (e.g., A, C, G, and T or U). Examples of enzymes or catalytic fragments thereof that may be optionally used include Klenow large fragment and T4 polymerase. At 5′ overhangs, the enzyme typically extends the recessed 3′ end on the opposing strand until it is flush with the 5′ end to produce a blunt end. At 3′ overhangs, the enzyme generally digests from the 3′ end up to and sometimes beyond the 5′ end of the opposing strand. If this digestion proceeds beyond the 5′ end of the opposing strand, the gap can be filled in by an enzyme having the same polymerase activity that is used for 5′ overhangs. The formation of blunt ends on double-stranded nucleic acids facilitates, for example, the attachment of adapters and subsequent amplification.

In some embodiments, nucleic acid populations are subjected to additional processing, such as the conversion of single-stranded nucleic acids to double-stranded nucleic acids and/or conversion of RNA to DNA (e.g., complementary DNA or cDNA). These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (e.g., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some embodiments, double-stranded nucleic acids with single-stranded overhangs in a sample after blunt-end formation are linked at both ends to adapters including barcodes, and the sequencing determines nucleic acid sequences as well as in-line barcodes introduced by the adapters. The blunt-end DNA molecules are optionally ligated to a blunt end of an at least partially double-stranded adapter (e.g., a Y-shaped or bell-shaped adapter). Alternatively, blunt ends of sample nucleic acids and adapters can be tailed with complementary nucleotides to facilitate ligation (for e.g., sticky-end ligation).

The nucleic acid sample is typically contacted with a sufficient number of adapters that there is a low probability (e.g., less than about 1 or 0.1%) that any two copies of the same nucleic acid receive the same combination of adapter barcodes from the adapters linked at both ends. The use of adapters in this manner may permit identification of families of nucleic acid sequences with the same start and stop points on a reference nucleic acid and linked to the same combination of barcodes. Such a family may represent sequences of amplification products of a nucleic acid in the sample before amplification. The sequences of family members can be compiled to derive consensus nucleotide(s) or a complete consensus sequence for a nucleic acid molecule in the original sample, as modified by blunt-end formation and adapter attachment. In other words, the nucleotide occupying a specified position of a nucleic acid in the sample can be determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand may be converted to their complements for purposes of compiling sequences to derive consensus nucleotide(s) or sequences. Some families include only a single member sequence. In this case, this sequence can be taken as the sequence of a nucleic acid in the sample before amplification. Alternatively, families with only a single member sequence can be eliminated from subsequent analysis.

Nucleotide variations (e.g., SNVs or indels) in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, for example, hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, and optionally which if any, include a reference nucleotide (e.g., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 8, 9, or 10 sequenced nucleic acids within the subset including the nucleotide variant or it can be a ratio, such as at least 0.5, 1, 2, 3, 4, 5, 10, 15, or 20, of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

Additional details regarding nucleic acid sequencing, including the formats and applications described herein, are also provided in, for example, Levy et al., Annual Review of Genomics and Human Genetics, 17: 95-115 (2016), Liu et al., J. of Biomedicine and Biotechnology, Volume 2012, Article ID 251364:1-11 (2012), Voelkerding et al., Clinical Chem., 55: 641-658 (2009), MacLean et al., Nature Rev. Microbiol., 7: 287-296 (2009), Astier et al., J Am Chem Soc., 128(5):1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, each of which is hereby incorporated by reference in its entirety.

IV. Computer Systems

Methods of the present disclosure can be implemented using, or with the aid of, computer systems. For example, such methods, which may comprise (a) adding a first subset of fragment size control molecules to the nucleic acid molecules in the sample of cell-free polynucleotides, thereby producing a first spike-in sample; (b) extracting nucleic acids from the first spike-in sample; (c) adding a second subset of fragment size control molecules to the extracted nucleic acids, thereby producing a second spike-in sample; (d) processing at least a subset of the second spike-in sample, thereby producing a processed sample, wherein the processing comprises partitioning, tagging, and/or amplifying the at least the subset of the second spike-in sample; (e) adding a third subset of fragment size control molecules to the processed sample, thereby producing a third spike-in sample; (f) enriching for at least a subset of the third spike-in sample, thereby producing an enriched sample; (g) adding a fourth subset of fragment size control molecules to at least a subset of the enriched sample, thereby producing a fourth spike-in sample; (h) sequencing the fourth spike-in sample to generate a plurality of sequence reads; (i) analyzing the plurality of sequence reads to generate a plurality of fragment size scores of the fragment size control molecules; and (j) comparing the plurality of fragment size scores with a plurality of fragment size thresholds, can be performed with a computer processor. In this embodiment, the system comprises components for adding fragment size control molecules, partitioning, amplifying, enriching and sequencing.

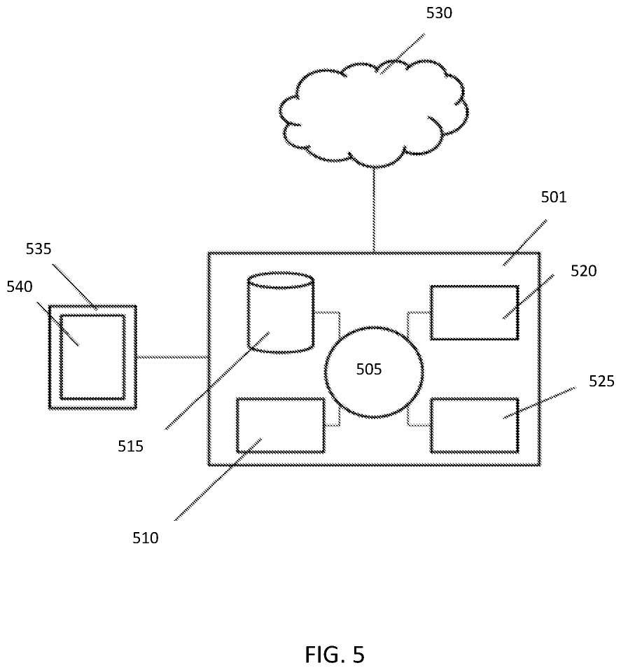

FIG. 5 shows a computer system 501 that is programmed or otherwise configured to implement the methods ofthe present disclosure. The computer system 501 can regulate various aspects sample preparation, sequencing, and/or analysis. In some examples, the computer system 501 is configured to perform sample preparation and sample analysis, including nucleic acid sequencing.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage, and/or electronic display adapters. The memory 510, storage unit 515, interface 520, and peripheral devices 525 are in communication with the CPU 505 through a communication network or bus (solid lines), such as a motherboard. The storage unit 515 can be a data storage unit (or data repository) for storing data. The computer system 501 can be operatively coupled to a computer network 530 with the aid of the communication interface 520. The computer network 530 can be the Internet, an internet and/or extranet, or an intranet and/or extranet that is in communication with the Internet. The computer network 530 in some cases is a telecommunication and/or data network. The computer network 530 can include one or more computer servers, which can enable distributed computing, such as cloud computing. The computer network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. Examples of operations performed by the CPU 505 can include fetch, decode, execute, and writeback.

The storage unit 515 can store files, such as drivers, libraries, and saved programs. The storage unit 515 can store programs generated by users and recorded sessions, as well as output(s) associated with the programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet. Data may be transferred from one location to another using, for example, a communication network or physical data transfer (e.g., using a hard drive, thumb drive, or other data storage mechanism).

The computer system 501 can communicate with one or more remote computer systems through the network 530. For embodiment, the computer system 501 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine-readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

In an aspect, the present disclosure provides a non-transitory computer-readable medium comprising computer-executable instructions which, when executed by at least one electronic processor, perform at least a portion of a method comprising: (a) adding a first subset of fragment size control molecules to the nucleic acid molecules in a sample of cell-free polynucleotides, thereby producing a first spike-in sample; (b) extracting nucleic acids from the first spike-in sample; (c) adding a second subset of fragment size control molecules to the nucleic acids, thereby producing a second spike-in sample; (d) processing at least a subset of the second spike-in sample, thereby producing a processed sample, wherein the processing comprises partitioning, tagging, and/or amplifying the at least the subset of the second spike-in sample; (e) adding a third subset of fragment size control molecules to the processed sample, thereby producing a third spike-in sample; (f) enriching for at least a subset of the third spike-in sample, thereby producing an enriched sample; (g) adding a fourth subset of fragment size control molecules to at least a subset of the enriched sample, thereby producing a fourth spike-in sample; (h) sequencing the fourth spike-in sample to generate a plurality of sequence reads; (i) analyzing the plurality of sequence reads to generate a plurality of fragment size scores of the fragment size control molecules; and (j) comparing the plurality of fragment size scores with a plurality of fragment size thresholds.

The code can be pre-compiled and configured for use with a machine have a processer adapted to execute the code or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display that comprises a user interface (UI) for providing, for example, one or more results of sample analysis. Examples of UIs include, without limitation, a graphical user interface (GUI) and web-based user interface.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

V. Applications
A. Cancer and Other Diseases

In some embodiments, the methods and systems disclosed herein may be used to identify customized or targeted therapies to treat a given disease or condition in patients based on the classification of a nucleic acid variant as being of somatic or germline origin. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

B. Therapies and Related Administration

In certain embodiments, the methods disclosed herein relate to identifying and administering customized therapies to patients given the status of a nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the test subject and/or patients who are receiving, or who have received, the same therapy as the test subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

1. -131. (canceled)

132. A method for analyzing nucleic acid molecules in a sample of polynucleotides, comprising:
a) adding a subset of fragment size control molecules to the nucleic acid molecules in the sample of polynucleotides, thereby producing a first spike-in sample;
b) extracting nucleic acids from the first spike-in sample, thereby producing extracted nucleic acids;
c) processing at least a subset of the extracted nucleic acids, thereby producing a processed sample,
wherein the processing is selected from the group consisting of (i) partitioning, (ii) tagging, and (iii) amplifying, at least a subset of the first spike-in sample;
d) enriching for at least a subset of the processed sample, whereby an enriched sample is produced;
e) sequencing at least a subset of the enriched sample to generate a plurality of sequence reads, wherein the plurality of sequence reads comprise sequence reads generated from the nucleic acid molecules in the sample of polynucleotides the fragment size control molecules; and
f) analyzing the plurality of sequence reads to generate a plurality of fragment size scores of the subset of fragment size control molecules.

133. The method of claim 132, further comprising, prior to c), adding a second subset of fragment size control molecules, thereby producing a second spike-in sample.

134. The method of claim 133, further comprising, prior to d), adding a third subset of fragment size control molecules, thereby producing a third spike-in sample.

135. The method of claim 134, further comprising, prior to e), adding a fourth subset of fragment size control molecules, thereby producing a fourth spike-in sample.

136. The method of claim 132, further comprising comparing the plurality of fragment size scores with a plurality of fragment size thresholds.

137. The method of claim 136, further comprising adjusting the analysis of nucleic acid molecules in the sample of polynucleotides based on the plurality of fragment size scores.

138. The method of claim 136, further comprising correcting for fragment size bias in the analysis of nucleic acid molecules in the sample of polynucleotides using the plurality of fragment size scores.

139. The method of claim 136, further comprising classifying the method as (i) being a success, if at least one of the plurality of fragment size scores is within a corresponding fragment size threshold of the plurality of fragment size thresholds;
or (ii) being unsuccessful, if at least one of the plurality of fragment size scores is not within the corresponding fragment size threshold of the plurality of fragment size thresholds.

140. The method of claim 132, wherein the subset of fragment size control molecules comprises a plurality of fragment size control molecules comprising a fragment size region.

141. The method of claim 140, wherein the fragment size control molecules further comprises an identifier region.

142. The method of claim 140, wherein the subset comprises at least one group of fragment size control molecules.

143. The method of claim 142, wherein the fragment size regions of the plurality of fragment size control molecules in a group are of same length.

144. The method of claim 141, wherein the identifier region is on one or both sides of the fragment size region.

145. The method of claim 141, wherein the identifier region comprises at least one molecular barcode.

146. The method of claim 141, wherein the identifier region comprises one or more primer binding sites.

147. The method of claim 140, wherein the fragment size regions of the fragment size control molecules in a group comprise the same oligonucleotide sequence.

148. The method of claim 140, wherein the fragment size regions of the fragment size control molecules in a first subset of fragment size control molecules comprises an oligonucleotide sequence distinguishable from the oligonucleotide sequence of the fragment size regions of the fragment size control molecules in a second subset of fragment size control molecules.

149. The method of claim 140, wherein length of the fragment size region is between 10 bp and 1000 bp.

150. The method of claim 142, wherein each of the groups of the fragment size control molecules in the subset is in non-equimolar concentration.

151. The method of claim 132, wherein the partitioning comprises partitioning the nucleic acid molecules of the at least the subset of the extracted nucleic acids into a plurality of partitioned sets, wherein the plurality of partitioned sets comprises nucleic acid molecules of the at least the subset of the extracted nucleic acids partitioned based on the epigenetic modification level of the nucleic acids.

152. The method of claim 132, wherein the tagging comprises attaching a set of tags to the nucleic acids to produce a population of tagged nucleic acids, wherein the tagged nucleic acids comprise one or more tags and the set of tags used in a first partitioned set of a plurality of partitioned sets resulting from the partitioning is different from the set of tags used in a second partitioned set of the plurality of partitioned sets.

153. The method of claim 152, wherein the set of tags is attached to the nucleic acids by ligation of adapters to the nucleic acids, wherein the adapters comprise one or more tags.

154. The method of claim 132, wherein concentration of the fragment size control molecules is between 1 attomole and 10 picomoles.

155. The method of claim 132, wherein the sample of polynucleotides is selected from the group consisting of (i) a sample of cell-free DNA, and (ii) a sample obtained from tissue.

156. The method of claim 155, wherein the sample of polynucleotides is between 1 ng and 500 ng.

157. The method of claim 132, wherein the fragment size control molecules are synthetic molecules.

158. The method of claim 132, wherein the fragment size control molecules are generated as amplicons by PCR amplification.
